(19) United States
(12) Patent Application Publication
(10) Pub. No.: US 2024/0198256 A1
(43) Pub. Date: Jun. 20, 2024

(54) Method for High Purity High Throughput Isolation of Extracellular Vesicles Using Size Exclusion Chromatography

(71) Applicant: Northeastern University, Boston, MA (US)

(72) Inventors: Alexander IVANOV, Newton, MA (US); Alan Zimmerman, Boston, MA (US)

(21) Appl. No.: 18/540,481

(22) Filed: Dec. 14, 2023

Related U.S. Application Data
(60) Provisional application No. 63/432,688, filed on Dec. 14, 2022.

(51) Int. Cl. B01D 15/38; B82Y 5/00

(52) U.S. Cl. CPC B01D 15/3847; B82Y 5/00

(57) ABSTRACT

The present technology provides straightforward, reproducible, scalable, and efficient multimode chromatography-based methods for the effective isolation and/or purification of nanoparticulate biomaterials from mixtures of biomaterials. The methods can be used to isolate and purify extracellular vesicles and other nanoparticulate biomaterials from plasma and other sources to provide high purity products. The methods can be used to diagnose medical conditions and to prepare pharmaceutical products.

### CROSS REFERENCE TO RELATED APPLICATIONS

This application claims the priority of U.S. Provisional Application No. 63/432,688 filed 14 Dec. 2022 and entitled “Method for High Purity High Throughput Isolation of Extracellular Vesicles Using Size Exclusion Chromatography”, the whole of which is hereby incorporated by reference.

### STATEMENT REGARDING FEDERALLY SPONSORED RESEARCH OR DEVELOPMENT

This invention was made with government support under Grant Number CA218500 awarded by the National Institutes of Health. The government has certain rights in the invention.

### BACKGROUND

Extracellular vesicles (EVs) are nanoscale membrane-enclosed entities that are shed from most cells within biological systems. EVs range in size, shape, density, and molecular composition, presumably reflecting the current state of the cell of origin when released into the extracellular space. Currently, EVs are classified into five major subpopulations: exomeres (<50 nm)¹, exosomes (˜30-150 nm), microvesicles (˜100-1000 nm)², apoptotic bodies (>1000 nm)³, and the recently termed supermeres⁴. While exomeres are not membrane enclosed, they still fall under the current definition of EVs5,6, which can also be termed extracellular vesicles and particles (EVPs)⁷. The Minimal

Information for Studies of Extracellular Vesicles 2018 (MISEV2018) is the current assembly of guidelines and considerations for researchers studying EVs^{3.}

EVs contain various biological molecules, including proteins, metabolites, and nucleic acids, that are specific to their cell of origin as well as their biogenesis⁸. Due to their critical roles in various biological processes such as immune responses and tumor progression while in circulation in the body, there is growing interest in exploiting EVs for liquid biopsy diagnostics as well as using their specificity for targeted therapeutics⁸⁻¹². However, it is a challenge to isolate pure EVs from other biomolecular species, such as free proteins, protein complexes, lipoprotein particles, and other macromolecular components of complex biological matrixes like blood plasma in a high throughput, time- and cost-efficient process.

Over the past decade, separation technologies such as size exclusion chromatography (SEC)^{13,14}, tangential flow filtration (TFF)^{15,16}, precipitation kits¹⁷, and asymmetric flow field flow fractionation (AF4)^{1,18} have made it possible to exploit the differences in EVs and to separate them from other highly abundant molecular and macromolecular species of complex biological specimens. These techniques appeared advantageous, increasing the effectiveness of isolation compared to the traditional isolation methods based on ultracentrifugation (UC) and density gradient centrifugation, which generate both protein and EV aggregates¹³ and are often low throughput. Additionally, each of these separation techniques has its own drawbacks as well. UC-based techniques, the current gold standard¹⁹, use differential centrifugation and ultracentrifugation at or above 100,000×g to isolate EVs based on density, which is a time-consuming and expensive process, and which does not appear to be easily scalable to process many samples at once²⁰. However, due to the density and size overlap with several types of lipoprotein particles, UC-based techniques are not adequate for sensitive molecular profiling, often resulting in contaminated samples^{21,22}. This is problematic for downstream EV characterization and proteomic analysis, as lipoprotein particles and their constituents can overload most analytical signals, making it nearly impossible to analyze intact EVs and their cargo with accuracy²¹. Particularly, apolipoprotein A-I, the main protein constituent in high-density lipoprotein (HDL), and apolipoprotein B100, the main protein in low-density lipoprotein (LDL)^{23,24} are of concern while working with EVs from plasma. Some reports have shown a purer EV sample can be obtained post-UC processing by washing with PBS several times, but will result in sample losses^{25,26}. Free plasma proteins are also of concern (serum albumin, IgGs alpha-2-macroglobulin, etc) having concentrations>1.5 orders of magnitude than apolipoproteins in plasma²⁷ which vastly limits the detection of lower abundant species such as EVs and their associated proteins.

### SUMMARY

The present technology provides straightforward, reproducible, scalable, and efficient multimode chromatography-based methods for the effective isolation and/or purification of nanoparticulate biomaterials from mixtures of biomaterials. In a preferred embodiment, the methods are used for depletion of plasma proteins and lipoprotein particles from samples containing extracellular vesicles (EVs) or extracellular vesicles and particles (EVPs) from plasma to provide higher purity products. The methods of the present technology are advantageous alternatives compared to conventional EV enrichment techniques such as ultracentrifugation and size exclusion chromatography.

An aspect of the present technology is a method of isolating and/or purifying a nanoparticulate biomaterial. The method includes the steps of: (a) providing a sample containing the nanoparticulate biomaterial suspended in an aqueous liquid and a multimodal chromatography resin; (b) contacting the sample with the multimodal chromatography resin for an incubation period; and (c) separating the nanoparticulate biomaterial from the multimodal chromatography resin, whereby the nanoparticulate biomaterial is isolated and/or purified from other components of the sample. The method can be carried out using the multimodal chromatography resin in suspension or configured as a column bed. Optionally, either the suspension mode or the column mode can be combined with another chromatography technique, such as size exclusion chromatography. The column mode also can be carried out using a sandwich column, having a first bed portion containing multimodal chromatography resin and a second bed portion containing a size exclusion chromatography resin, and the first bed portion is preferably disposed higher in the column than the second bed portion. A multilayer column format also can be used, in which two or more layers of different chromatography resins are stacked one over the other. One or more of the layers contain or consist of a multimodal chromatography resin, and one or more of the other layers contain or consist of a unimodal chromatography resin operating on a different principle. Preferably, the different chromatography resins have different separation mechanisms, although the same separation mechanism also can be repeated in different layers of the column. The nanoparticulate biomaterial to be isolated and/or purified can be, for example, extracellular vesicles and particles (EVP), viruses, viral vectors, pseudoviruses, virus-like particles, polymeric nanoparticles, lipidic nanoparticles, artificial lipid membrane vesicles, or nanoparticulate drug carriers. Viral vectors such as lentiviral vectors and adenoviral or adeno-associated viral vectors can be isolated and/or purified using the present methods, for example. Any type of virus, virus-like particles, or pseudoviruses can be isolated and/or purified using the present methods. Vaccines or other products containing polymeric nanoparticles, polymer-encapsulated nanoparticles (including viral vectors), or lipid-associated or lipid membrane encapsulated particles or vesicles can be isolated and/or purified using the present methods, The sample can contain these nanoparticulate materials together with undesired smaller particles and/or molecules, such as plasma proteins; lipoprotein particles; immunoglobulins; cells or cell fragments; molecular components such as proteins, nucleic acids, polysaccharides, lipids, or complexes thereof; metabolites; small molecules including pharmaceutical agents; and viruses or portions thereof, which can be removed from the desired nanoparticles using the methods described herein.

Another aspect of the present technology is a chromatography column configured for performing the method described above. For example, the chromatography column can contain one or more multimodal chromatography resins, and in some embodiments also can contain one or more additional chromatography resins, particularly unimodal chromatography resins that use a single separation principle, such as one or more size exclusion chromatography resins. Examples of multimodal chromatography resins include CaptoCore 400 (molecular weight cutoff>400 kDa)and CaptoCore 700 (molecular weight cutof>700 kDa), and examples of size exclusion chromatography resins include Sepharose CL-2B (fractionation range 70-40,000 kDa), Sephacryl S500 (fractionation range 40-20,000 kDa), and Sephacryl S1000 (fractionation range 500-100,000 kDa). In a preferred embodiment, the chromatography column is configured as a sandwich column, having a bed of multimodal chromatography resin positioned above a bed of size exclusion chromatography resin, or configured as a multilayered column having greater than two different resin layers, at least one of which contains or consists of a multimodal chromatography resin. The multimodal chromatography resin retards the migration of undesired molecular species and other undesired biomaterials while the desired nanoparticulate biomaterial advances down the column, with further separation by size carried out by the size exclusion resin.

Yet another aspect of the present technology is a kit for isolating and/or purifying a nanoparticulate biomaterial. The kit includes a multimodal chromatography resin or a column comprising a multimodal chromatography resin and optionally another type of chromatography resin, such as an SEC resin. The kit further includes instructions for carrying out the method of isolating and/or purifying a nanoparticulate biomaterial as described above.

Still another aspect of the technology is a method of diagnosing a disease or medical condition. The method includes the steps of: (a) providing a kit as described above and a sample from a subject suspected of having the disease or medical condition; (b) performing the method of isolating and/or purifying a nanoparticulate biomaterial as described above, whereby an isolated and/or purified nanoparticulate biomaterial is obtained; and (c) analyzing the isolated and/or purified nanoparticulate biomaterial to diagnose the disease or medical condition. Analysis of the nanoparticulate biomaterial can include, for example, determining its molecular composition, size, structure, or the presence or absence of any of certain biomarkers whose presence or absence are characteristic of the disease or medical condition. Examples of diseases or medical conditions that can be diagnosed using this method include viral infections, cancers including prostate, brain, and breast cancer; neurodegenerative diseases including Alzheimer's disease and Parkinson's disease, and cardiovascular diseases. In some cases, diagnosis by this method can be followed by an appropriate treatment for the disease or medical condition.

As used herein, the term “nanoparticulate biomaterial” refers to a particle or particle-like material of biological origin, or of synthetic origin but containing one or more natural or non-naturally occurring biomolecules. Examples include extracellular vesicles; fragments of cells or cellular structures; viruses, subviral particles, viral vectors, or pseudoviruses; complexes of proteins, nucleic acids, polysaccharides, or lipids, or any combination thereof; natural or synthetic antibodies and fragments thereof; polymeric nanoparticles and nanoparticles coated with polymers; natural and artificial lipid vesicles and lipid membrane-encapsulated structures; pharmaceutical delivery vehicles, and subpopulations and combinations of any of the foregoing. In preferred embodiments, nanoparticulate biomaterials have an average size from about 1 nm to about 1000 nm, or less than 500 nm, or less than about 400 nm, or less than about 200 nm, or less than about 100 nm.

As used herein, the term “extracellular vesicles” (EV) is used synonymously with the term “extracellular vesicles and particles” (EVP). That is, unless stated to the contrary, reference to EV is intended to also include EVP. EVs can include exomeres and supermeres (having a size of less than 50 nm), exosomes (having a size from about 30nm to about 150 nm), and microvesicles (having a size from about 100 nm to about 1000 nm). Apoptotic bodies (having a size of greater than about 1000 nm) also can be considered within the class of extracellular vesicles.

As used herein, the “size” of a particle refers to its diameter, when the particle has a substantially spherical geometry. If the particle has an irregular shape, the size of the particle refers to the diameter of a minimal sphere that can contain the particle.

As used herein, the term “multimodal chromatography” refers to any type of chromatography used to separate biomaterials that involves two or more separation principles simultaneously. For example, multimodal chromatography can utilize a multimodal chromatography resin containing porous particles or beads having a diameter in the micron range and having a functional group found selectively or exclusively inside the particles but not on the outside of the particles. An example is CaptoCore by Cytiva, available as CaptoCore 400 (pore radius 19 nm) and CaptoCore700 (pore radius 50 nm), which has cationic functional groups inside the resin particles that can perform anion exchange chromatography on molecules or structures having a size that allows their entry into the pores.

As used herein, the term “multiphase column” or “multiphase chromatography column” refers to a column chromatography format in which the column contains two or more different chromatographic materials, each performing a separation of biomolecules or biomaterial structures using a different separation principle. When the two or more different chromatographic materials are segregated into different regions of the column, one above the other, the column is referred to as a “sandwich column”.

The present technology also can be summarized with the following list of features.

1. A method of isolating and/or purifying a nanoparticulate biomaterial, the method comprising the steps of:
(a) providing a sample comprising the nanoparticulate biomaterial and a multimodal chromatography resin, wherein the sample comprises said nanoparticulate biomaterial suspended in an aqueous liquid; and
(b) contacting the sample with the multimodal chromatography resin for an incubation period, and;
(c) separating the nanoparticulate biomaterial from the multimodal chromatography resin, whereby the nanoparticulate biomaterial is isolated and/or purified from other components of the sample.

2. The method of feature 1, wherein the nanoparticulate biomaterial is selected from the group consisting of extracellular vesicles and particles (EVP), viruses, viral vectors, pseudoviruses, virus-like particles, polymeric nanoparticles, lipidic nanoparticles, artificial lipid membrane vesicles, and nanoparticulate drug carriers.

3. The method of feature 2, wherein the nanoparticulate biomaterial is EVP comprising exomeres, supermeres, exosomes, and/or microvesicles.

4 The method of any of the preceding features, wherein the nanoparticulate biomaterial has an average particle size in a range from about 50 nm to about 100 nm, or from about 100 nm to about 150 nm, or from about 100 nm to about 200 nm, about 200 nm or less, from about 200 nm to about 1000 nm, or more than 1000 nm.

5. The method of any of the preceding features, wherein the sample is a biological fluid; plasma; interstitial fluid; cerebrospinal fluid; saliva; cell culture medium; homogenized biological cells, tissue, or extracellular matrix; a pharmaceutical composition; a vaccine composition; or aerosol droplets or breath condensate; tear fluid; tissue aspirate, sputum; nasal fluid; lavage; artificially prepared emulsion, micelles, lipid droplets, or liposomes; or a tissue homogenate from a mammal, plant, mushroom, or alga.

6. The method of any of the preceding features, wherein the multimodal chromatography resin comprises porous resin particles having one or more functional groups selectively disposed inside the porous resin particles.

7. The method of feature 6, wherein the functional groups are selected from ionic groups, hydrophobic groups, hydrophilic groups, amphiphilic groups, polar uncharged groups, non-polar groups, affinity-based groups, octylamine, and N-benzyl, N-methyl ethanolamine.

8. The method of feature 6 or 7, wherein the multimodal chromatography resin is capable of separating said nanoparticulate biomaterial from other components of the sample by at least a first mode and a second mode, wherein the first mode is size exclusion chromatography, and wherein the second mode is different from size exclusion chromatography.

9. The method of feature 8, wherein the second mode is ion exchange chromatography, hydrophobic interaction chromatography, reversed phase chromatography, or affinity chromatography.

10. The method of feature 9, wherein the ion exchange chromatography is anion exchange or cation exchange chromatography.

11. The method of any of features 6-10, wherein the porous resin particles comprise pores having an average size of about 19 nm, about 50 nm, or about 72 nm.

12. The method of any of the preceding features, wherein the step of contacting is performed using a suspension of particles of said multimodal chromatography resin in an aqueous medium.

13. The method of feature 12, wherein the suspension is mixed during the incubation period.

14. The method of any of the preceding features, wherein the incubation period is long enough to attain binding equilibrium of undesired components of the sample with the functional groups inside the porous resin particles of the multimodal chromatography resin.

15. The method of any of features 12-14, wherein the step of separating includes centrifugation or filtration of the suspension and harvesting the isolated and/or purified nanoparticulate biomaterial from the supernatant or filtrate.

16. The method of any of features 1-11, wherein the step of contacting is performed using a bed of said multimodal chromatography resin, the bed disposed in a chromatography column, and wherein the step of separating is performed by eluting fractions from the column, some of said fractions comprising the isolated and/or purified nanoparticulate biomaterial.

17. The method of feature 16, wherein the chromatography column is a sandwich column comprising the bed of said multimodal chromatography resin and a second bed comprising a second chromatography resin, wherein the multimodal chromatography resin and the second chromatography resin are capable of different modes of separation.

18. The method of feature 17, wherein the multimodal chromatography resin performs size exclusion and anion exchange and the second chromatography resin performs size exclusion.

19. The method of feature 17, wherein the multimodal chromatography resin is disposed above the second chromatography resin in the column.

20. The method of feature 16, wherein the chromatography column is a multilayer column comprising the bed (layer) of said multimodal chromatography resin and at least two other beds (layers) comprising a other chromatography resins, wherein the multimodal chromatography resin and the other chromatography resins are capable of different two or more modes of separation.

21. The method of any of the preceding features, wherein the method does not comprise ultracentrifugation.

22. The method of any of the preceding features, wherein the nanoparticulate biomaterial is separated from other biomaterial present in the sample.

23. The method of feature 22, wherein the other biomaterial is protein or other particulate material.

24. The method of any or the preceding features, wherein the nanoparticulate biomaterial is fractionated to yield one or more isolated and/or purified subpopulations.

25. The method of feature 24, wherein the nanoparticulate biomaterial that is isolated and/or purified is EVP, and the other biomaterial is plasma protein, albumin, immunoglobulin, alpha-2 macroglobulin, apolipoprotein B100, apolipoprotein A1, haptoglobin, serotransferrin, and/or alpha-1-antitrypsin.

26. The method of feature 23 or 25, wherein the isolated and/or purified EVP are enriched in CD9, annexin A2, and/or 14-3-3 zeta/delta compared to the sample.

27. The method of any of the preceding features, further comprising analyzing the separated nanoparticulate biomaterial using a method selected from TEM, BCA, WB, TRPS, nFCM, or nLC-MS/MS.

28. A chromatography column configured for performing the method of any of the preceding features.

29. The chromatography column of feature 28, comprising a multimodal chromatography resin and a size exclusion chromatography resin, and optionally one or more other chromatography resins, wherein the chromatography column is configured as a sandwich column or a multilayered column.

30. A kit for isolating and/or purifying a nanoparticulate biomaterial, the kit comprising:
a multimodal chromatography resin or a column comprising a multimodal chromatography resin; and instructions for carrying out the method of any of features 1-26.

31. A method of diagnosing a disease or medical condition, the method comprising the steps of:
(a) providing the kit of feature 27 or the chromatography column of feature 25 or 26 and a sample from a subject suspected of having the disease or medical condition;
(b) performing the method of any of features 1-24, whereby an isolated and/or purified nanoparticulate biomaterial is obtained; and
(c) analyzing the isolated and/or purified nanoparticulate biomaterial to diagnose the disease or medical condition.

32. The method of feature 31, further comprising:
(d) treating the disease or medical condition.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1N illustrate an initial assessment of CaptoCore for EV Isolation. FIG. 1A shows a diagram of the general workflow for isolation and characterization methods of blood plasma derived EVs. FIGS. 1B-1I show comparisons of CaptoCore 400 and CaptoCore 700 pore sizes by SEM imaging. The size distribution varies slightly between individual beads within CaptoCore 400 (1B) and 700 (1F) with the median bead diameter of ˜90 μm. Single beads of CaptoCore 400 and 700 were individually imaged (FIG. 1C and FIG. 1G). Inset images reveal the pore size differences between the two types of beads (1D and 1H), which is further exemplified by zooming in to the nanoscale (1E and 1I). FIG. 1J shows protein binding capacity of CaptoCore 400 and 700 measured using a BCA assay. FIG. 1K shows EV recovery rate measured by TRPS, using starting material equivalent to 0.1 μL of undiluted plasma. CaptoCore 700 had a lower EV recovery rate, but a higher protein binding capacity. CaptoCore 400 had a higher EV recovery rate, but a lower protein binding capacity. FIGS. 1L-1N show elution profiles of EVs and free plasma proteins using CL-2B (1L), SW 400 (1M), and SW 700 (1N). Fractions were collected in 200 μL increments.

FIG. 2A shows intact EV morphology and size distribution assessed by TEM imaging and nFCM. EVs from all isolation methods show cupping, a classic type of EV morphology from the sample preparation process. Their size was approximately <200 nm in diameter. Scale bars at the top right of images correlate to the images and scale bars at the bottom left of images correlate to the inset panels. FIG. 2B shows particle counts across a 30 sec acquisition determined by nFCM. Particle counts correlate to expected concentrations for each isolation method, with Sus 400 generating the most counts within the gated area over a 30 sec acquisition time and a UC-generated EV isolate displaying the least amount of counts within the same gated area over the same acquisition time. In FIG. 2Bii median particle size distributions are shown as determined for the same gated area across the same acquisition time, revealing similar median sizes across all methods. CaptoCore 700 generated samples had a slight increase in their size distributions, presumably due to smaller particles becoming trapped inside the larger pores. UC also had a slight increase in determined size, presumably to due the loss of smaller particles during the isolation process. FIG. 2Biii shows a histogram of all isolation methods for comparison.

In FIGS. 3A-3D, immunoaffinity-based assays were employed to determine intact particle concentration as well as characterization of specific protein markers. FIG. 3A shows particle concentration determined by immunoaffinity-based interferometry, demonstrating a similar trend compared to nFCM, with the Sus 400-generated sample yielding the highest concentration of tagged and counted particles. The inset illustrates that the SW 700-generated EV isolate yielded a lower concentration than other isolation methods; however, it had more detectable particles than the negative control, dPBS. FIG. 3B shows images of the fluorescently tagged particles. Noticeably, UC-generated EVs aggregated together, whereas EVs prepared by all other isolation methods did not. FIG. 3C shows western blots of proteins of interest to determine whether plasma depletion was successful while retaining EVs. Apolipoprotein A1 and IgG heavy chain show the greatest depletion, with little to no bands detected. Albumin and apolipoprotein B-100 had detectable bands; however, CaptoCore-based methods showed weaker bands compared to a conventional SEC-generated EV isolate. CD9 was detectable with each isolation method. FIG. 3D shows silver stained SDS-PAGE gels of total protein from each isolation method, indicating CaptoCore-based samples, especially CaptoCore 700-based samples, had lower protein amounts. Sample loading volumes for both 3C and 3D were as follows from left to right: 1 μL SEC, 10 μL SEC, 1 μL Sus 400, 1 μL Sus 700, 10 UL SW 400, 10 μL SW 700, 0.01 μL plasma.

FIGS. 4A-4I show proteomic analysis of human blood plasma-derived EV-related proteins and associated plasma proteins. FIG. 4A is a heat map showing all proteins detected during bottom-up mass spectrometry analysis for each isolation method. Insert 4Ai shows the most highly abundant plasma proteins clustered together. Insert 4Aii shows a cluster of EV-related proteins, including 14-3-3 zeta/delta, CD9, and GAPDH. FIG. 4B is an extracted heat map of EV-related proteins. FIG. 4C is an extracted heat map of all apolipoproteins detected. FIG. 4D is an extracted heat map of the top plasma proteins. All scales are log2 abundance. FIG. 4E is a Venn diagram comparing the number of identified proteins obtained using traditional isolation methods (SEC ad UC) to the number of proteins obtained using CaptoCore-based Sus isolation methods. FIG. 4F is a Venn diagram comparing the number of identified proteins obtained using traditional isolation methods (SEC and UC) compared to CaptoCore-based SW isolation methods. FIG. 4G is a Venn diagram comparing the number of identified proteins obtained using the CaptoCore-based methods, Sus and SW. FIG. 4H is a PCA plot illustrating that UC-based EV isolates cluster further away from all other methods. FIG. 4I is a GO Term analysis of selected terms of interest showing the percentage of identified proteins within each method.

FIGS. 5A-5E show analysis of binding kinetics of BSA and IgG protein standards (5A, 5B) and plasma (5C, 5D) binding to CaptoCore 700, measured using the BCA assay. 90% of standard protein bound after 10 mins (for 2 mg and 6 mg) and 90% of plasma protein was bound after 10 mins (13×, 20×, and 40× plasma). Equilibrium was reached at 30 mins for both standard proteins and plasma proteins. FIG. 5E shows silver stained SDS-PAGE, revealing the depletion of proteins from plasma over time.

FIGS. 6A-6E show determination of binding capacity of CaptoCore 400 in suspension mode, which was found to be 17.9±0.1 mg protein standard per 1 mL of CaptoCore 400, and 19.4±0.4 mg plasma protein per 1 mL of CaptoCore 400, as measured using the BCA assay (6A-6D). FIG. 6E shows Coomassie stained SDS-PAGE gels. FIGS. 7B and 7D show residual proteins normalized to the starting protein amount.

FIGS. 7A-7E show determination of binding capacity of CaptoCore 700 in suspension mode, which was found to be 20.1±0.5 mg with BSA and IgG protein standard per 1 mL of CaptoCore 700, and 23.75±0.5 mg plasma protein per 1 mL of CaptoCore 700, as measured using the BCA assay (7A-7D). FIG. 7E shows silver stained SDS-PAGE gels. FIGS. 7B and 7D show residual proteins normalized to the starting protein amount.

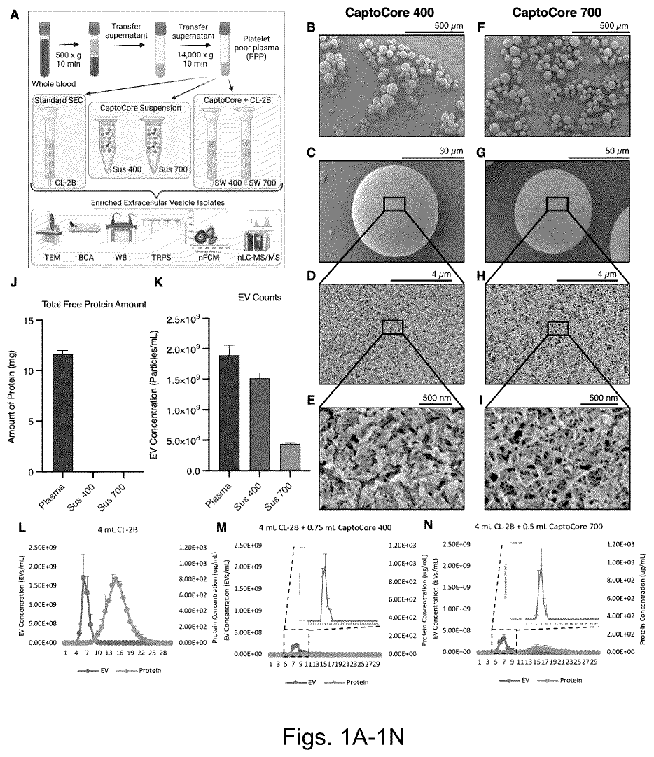

FIG. 8 shows SDS-PAGE confirming separation of free plasma proteins from EVs. The results show that C700 (Sus 700) removed protein contamination, while C400 (Sus 400) was unable to remove all protein contamination (indicated with an oval) in suspension mode (corresponding to FIG. 1J). Starting material was equivalent to 0.1 μL undiluted plasma.

FIGS. 9A-9F show results of optimization of sandwich column geometry for CaptoCore 400 (9A-9B) and CaptoCore 700 (9C-9D). SDS-PAGE of SW 400 (9E) and SW 700 (9F) show the majority of plasma proteins eluting later, giving EVs the opportunity to elute without interference.

FIG. 10 shows a representative image of dPBS used as a negative control to illustrate no EV-like features were detected in the buffer used for sample processing.

FIG. 11 shows nanoflow cytometry gating strategy. Dulbecco's phosphate-buffered saline was used as a negative control to determine the background noise intensity. Polystyrene beads ranging from 80 to 500 nm were used to determine the violet side scatter width and height. To determine EV count and size distribution, EVs were gated between the noise and ˜10⁵ violet side scatter height, called EV Singlet. Another gate was used that included the noise, called EV Singlet Noise, to determine the scattering cross section.

FIGS. 12A-12F show the size distributions of EVs obtained by the indicated isolation methods as measured by TRPS. FIG. 12A shows results using a 4 mL CL-2B SEC column. FIG. 12B shows results using CaptoCore 400 in Sus mode. FIG. 12C shows results obtained using CaptoCore 700 in Sus mode. FIG. 12D shows results using a column containing 4 mL CL-2B+0.75 mL CaptoCore 400 in sandwich mode. FIG. 12E shows results obtained using a column containing 4 mL CL-2B+0.5 mL CaptoCore 700 in sandwich mode. FIG. 12E shows results obtained for a UC-generated sample. The sizes and concentrations of EVs are approximately the same for each method, indicating consistency among methods.

FIG. 13 shows scattering cross section for EV isolated by the indicated methods, determined by nanoflow cytometry measurements using the EV Singlet Noise gate. Background noise was determined to be constant during sample acquisition.

FIG. 14 shows replicate immunoaffinity-based interferometry images of the indicated samples. Images were taken from different replicate spots, showing constant immunoaffinity for each tagged tetraspanin. Images correspond to FIG. 3B.

FIGS. 15A-15E show EVs (measured using TRPS) and proteins (measured with a BCA assay) in each fraction from SEC columns containing (15A) 2 mL (15B) 3 mL (15C) 4 mL and (15D) 4 mL of CL-2B with a filter. FIG. 15E shows SDS-PAGE of fractions obtained from 4 mL CL-2B, illustrating protein separation from the EV-containing fractions. Starting material was equivalent to 0.1 μL undiluted plasma.

FIGS. 16A-16D show binding capacity determination using protein standards (FIGS. 16A, 16B) and plasma (16C, 16D) of CaptoCore 700 resin in column format.

### DETAILED DESCRIPTION

Extracellular vesicles (EVs) play a pivotal role in various biological pathways, such as immune responses and the progression of diseases, including cancer. However, it is challenging to isolate EVs at high purity from blood plasma and other biofluids due to their low abundance compared to more predominant biomolecular species such as lipoprotein particles and free protein complexes. Ultracentrifugation-based EV isolation, the current gold standard technique, cannot overcome this challenge due to similar densities of such species. The present technology provides several novel approaches to enrich EVs from plasma while depleting contaminating molecular species using multimode chromatography-based strategies. Using the present technology, on average, 716±68 proteins and 1054±35 proteins were identified in EV isolates from 100 μL of plasma using multimode chromatography- and ultracentrifugation-based techniques, respectively. The developed methods resulted in similar purity of EV isolates, providing significant advantages in simplicity, throughput, scalability, and applicability for various downstream analytical and potential clinical applications.

To alleviate the limitations of centrifugation techniques, the present inventors have exploited the properties of size exclusion chromatography (SEC), which has been used for isolation of EVs from plasma^{28,29}, to separate various biomolecules and macromolecular protein complexes based on their hydrodynamic diameter¹³. There are multiple advantages of SEC, including high throughput, cost efficient, ease of use, scalability, tolerance to various mobile phases, and the effectiveness of EV enrichment¹³. While SEC is generally considered a low-resolution technique, it provides sufficient separation power to resolve the chromatographic peaks corresponding to EVs and other large MDa-scale macromolecular assembles from smaller molecular species, such as high abundance proteins and protein complexes that elute later. However, the chromatographic peaks are usually not baseline resolved by the most common gravity flow- or pressure drop-driven separations, using cartridge columns packed with only Sepharose CL-2B or similar SEC beads^{13,29.}

In order to obtain higher resolution by SEC, the performance of two types of CaptoCore resins (Cytiva) was evaluated. CaptoCore is a multimode, fully porous resin, which demonstrates both SEC and anion exchange (AEX) chromatography modes of separation at the same time. Negatively charged molecular species that are less than the molecular the weight cut-off (MWCO) of the two CaptoCore resins (i.e., 400 kDa or 700 kDa) become trapped on the AEX surface inside the beads by interacting with the positively charged octylamine functional group^{30,31}. Meanwhile, the SEC mode enables the separation of EVs, which are generally larger than the MWCOs of the beads (i.e., the exclusion limit), from other smaller molecular and macromolecular species³⁰. CaptoCore has been used in a number of ways to purify complex biological samples. For example, CaptoCore has been used to isolate virus particles as an alternative to UC for a more time-efficient isolation method^{30,32}. There are multiple characteristics shared between

EVs and virus particles or other nanoparticulate biomaterials, such as size, shape, and chemical composition, which all influence cellular uptake, and hence, the possibility for particular isolation approaches³³. More recently, CaptoCore has been employed to remove unlabeled dye from EV samples intended to be analyzed by flow cytometry, named EV-Clean³⁴.

The EV isolation performance of conventional SEC columns packed with Sepharose CL-2B beads was evaluated. This is a common SEC stationary phase for EV isolation,^{13,35} The optimal column geometry for EV enrichment was determined. Then, the two types of CaptoCore resins were investigated alone for their performance in plasma-derived EV enrichment, which was named Suspension mode (Sus). Additionally, to further improve upon a traditional SEC method, the inventors employed the use of CaptoCore by layering the resin on top of CL-2B beads in a biphasic chromatographic form the inventors named a “sandwich column” (SW column) to allow for better resolution between EVs and free plasma proteins (FIG. 1A). The purity, size distribution, morphology, concentration, and proteomic molecular profiles of the resulting EV isolates were characterized using transmission electron microscopy (TEM), tunable resistive pulse sensing (TRPS), immunoaffinity-based interferometry, gel electrophoresis, the western blotting, nanoflow cytometry (nFCM), and proteomic analysis by nanoflow liquid chromatography coupled with tandem mass spectrometry (nLC-MS/MS). Results show that successful EV isolation was achieved with substantial depletion of free plasma proteins using both Sus and SW modes of separation, saving overall sample processing time, with similar EV enrichment to the traditional SEC- and UC-based methods. The impact of the different pore sizes of the two types of CaptoCore beads on the isolation of EVs was also investigated. The inventors determined that the EV recovery rate was higher using CaptoCore 400 beads, specifically Sus 400, without the use of a SEC column. However, this resin was less efficient in depleting free plasma proteins due to the smaller pore size not trapping as many free plasma proteins and protein complexes. In contrast, the CaptoCore 700 resin resulted in a lower EV recovery rate, but in better efficiency of free plasma protein depletion, which ultimately led to a higher purity EV isolate, specifically from the developed SW 700 column.

The novel features of the present technology include the following: (i) use of porous multimodal chromatography beads which allows for lipoprotein particles, large protein complexes, nucleic acids, and other nanoscale structures to interact with the chemical functional groups (e.g., octylamine ligand core for anion exchange and hydrophobic interaction), leading to higher purity and higher concentration than obtained by size exclusion or centrifugation alone; (ii) use of sandwich or multilayered columns offers the benefits of two chromatography resins layered on top of each other or in layers to increase separation effectiveness for superior removal of contaminants; and (iii) faster, more efficient separation and purification by combining different separation modalities in a single column run.

The present technology offers several advantages or improvements over previous technologies, including: (i) use of sandwich columns which are more cost-effective compared to ultracentrifugation; (ii) high throughput compared to ultracentrifugation; (iii) non-labor-intensive processing with no specialized training necessary; (iv) higher purity and greater efficiency in isolation of nanoparticulate biomaterials; and (v) widely applicable methodology.

Uses of the present technology include: (i) enrichment of EVs from complex matrices; (ii) enrichment of viruses and other nanoscale particles from complex matrices; (iii) pre-treatment of EVs before isolation of EV subpopulations; (iv) isolation and purification of a variety of biomaterials; and (iv) purification and finishing of pharmaceutical compositions and drug delivery formulations.

### EXAMPLES

### Example 1

### Reagents and Methods

Ten×phosphate-buffered saline (PBS) (Cat. #BP399-1), Pierce™ BCA Protein Assay Kits (Cat. #23227), 10×reducing agent (Cat. #NP0004), were obtained from Thermo Fisher Scientific (Waltham, MA). Dulbecco's phosphate-buffered saline (dPBS) (Cat. #D8537) was obtained from Millipore Sigma (Burlington, MA). Chromatography resins, Sepharose CL-2B, CaptoCore 400, CaptoCore 700, and PlusOne Silver Staining Kit (Cat. #17115001) were obtained from Cytina Life Sciences (Marlborough, MA, USA) lgG (Cat. #18640) and BSA (Cat. #A2153) were obtained from Millipore Sigma (Burlington,

MA). Four×lithium dodecyl sulfate (LDS) sample buffer (Cat. #NP0007), 10×dithiothreitol (DTT) (Cat. #B0009), and 4-12% Bis-Tris gel (Cat. #NP0323) were obtained from Invitrogen (Waltahm, MA). Pierce Silver Stain Kit (Cat. #24612) were obtained from Thermo Scientific (Waltham, MA). Mouse monoclonal anti-human CD9 antibody (Cat. #10626D, clone TS9, dilution 1: 750) was purchased from Invitrogen. Antibodies [mouse monoclonal anti-human ALB antibody (Cat. #sc-271605, clone F-10, dilution 1:500), mouse monoclonal anti-human IgG antibody (Cat. #sc-69786, clone 3E8, 1:1000), mouse monoclonal anti-human apoA-I antibody (Cat. #sc-376818, clone B-10, dilution 1:250), mouse monoclonal anti-human apoB-100 antibody (Cat. #sc-13538, clone C1.4, dilution 1:500)] were purchased from Santa Cruz Biotechnology, Inc. (Dallas, TX).

Horseradish peroxidase (HRP)-conjugated mouse IgG K light chain binding protein (Cat. #sc-516102, dilution 1:10000) was also purchased from Santa Cruz Biotechnology, Inc. SuperSignal West Femto Maximum Sensitivity Substrate (Cat. #34096) was purchased from Thermo Scientific (Waltham, MA).

### Plasma Preparation

Cell- and platelet-free anticoagulated with EDTA plasma was collected from self-declared healthy male donors and pooled together (IRB #2001P000591 (BIDMC) and IRB #17-12-14 (NU)). One mL plasma aliquots were stored at −80° C. until use. Prior to EV isolation, the plasma was thawed at RT and spun at 14,000×g for 10 mins (Centrifuge 5424 R, Eppendorf; Hamburg, Germany), and the lower plasma layer was collected; this is referred to as platelet-poor plasma (PPP).

### Scanning Electron Microscopy (SEM) Imaging

For SEM, CaptoCore beads were placed on a specimen holder using small pieces of conductive adhesive (2×2 mm) and visualized using a Hitachi S-4700 FE-SEM.

### Transmission Electron Microscopy (TEM) Imaging

5 μL of the EV samples were placed on parafilm paper (Bemis Company, Inc, Neenah, WI). Carbon-coated copper grids (Electron Microscopy Sciences, Hatfield, PA) were placed on top of the samples and incubated for 10 min at room temperature. The grids with samples were then washed once in 1×PBS and negatively stained using 2% uranyl acetate for 1 min. Samples were visualized on a JEOL 1400 electron microscope outfitted with an Orius SC1000 CCD camera (Gatan, Inc. Pleasanton, CA).

### NanoFlow Cytometry

The CytoFLEX S used was the 13 color, 4-laser V-B-YG-R model equipped with 405 nm, 488 nm, 561 nm, and 638 nm lasers (C09766, Beckman Coulter, Brea, CA). The modifications of the instrument for nanoflow cytometry are those described in Brittain et al⁵¹. In brief, the 450/45 filter and the 405/10 filter were swapped (eliminating the PB450 channel). The detector configuration in the CytExpert software was updated to reflect this. VSSC-Height was used as the trigger channel and the threshold level was manually set based on instrument noise. The threshold level was checked by using 80 nm PS NIST beads (64008, Polysciences, Inc., Warrington, PA) at concentration of 1×105/mL. Prior to running beads or samples, the instrument was cleaned using FlowClean Cleaning Agent (A64669, Beckman Coulter, Brea, CA) and DI water to flush out debris. This step was repeated as necessary to reduce background contamination.

### Tunable Resistive Pulse Sensing (TRPS)

The concentration and size of sample particles were measured using TRPS (qNano Gold; iZon Science Ltd.; Christchurch, New Zealand). Purified plasma EVs were diluted in 1×PBS with size and concentration to be determined using a NP150 nanopore. Calibration was performed using CPC200 beads, diluted in 1×PBS, and measured at identical settings of the samples. Analyses of the particle concentration and size distributions were performed using Izon Control Suite V3.3 software.

### Bicinchoninic Acid Assay (BCA Assay)

Bicinchoninic acid assays were performed using Pierce™ BCA Protein Assay Kits (Cat. #23227, Thermo Fisher) and following the manufacturer's protocol. 2 μL of sample or protein standard were mixed with 150 μL of BCA reagent in a 96 well polystyrene plate (Cat. #15041, Thermo Fisher), sealed, and incubated for 2 hours at 37° C. The absorbance was measured at 562 nm.

### Interferometry

The abundance of EVs was measured by interferometric imaging using the ExoView R200 and human tetraspanin kit (NanoView Biosciences Inc.). All samples were diluted 25×using incubation buffer and the assay was run according to the manufacturer's protocol. Briefly, 50 μL of the diluted samples were incubated on the ExoView chip overnight (16 h) at room temperature. Afterwards, the sample incubation chips were washed and stained for 1 h with an antibody cocktail provided in the kit that consists of anti-CD81 CF555 (JS-81), anti-CD63 CF647 (H5C6), and anti-CD9 CF488 (HI9a). After staining, the chips were washed, dried and imaged with the R200 reader. NanoViewer Analysis software 3.04 was used to calculate the particle count for each capture spot. Interferometry measurements were performed using an established protocol⁴⁴.

Gel Electrophoresis

Samples were mixed with 5 μL of 4×LDS sample buffer (Cat. #NP0007, Invitrogen) and 2 μL of 10×DTT (Cat. #B0009, Invitrogen). Samples were heated at 70° C. for 10 min and loaded on a 4-12% Bis-Tris gel (Cat. #NP0323, Invitrogen). The gel ran at 200 V for 35 mins and stained with Pierce Silver Stain Kit (Cat. #24612, Thermo Scientific).

### Western Blot

Five μL of each sample as well as a reference PPP was denatured and then loaded onto the 4-12% Bis-Tris gel (Cat. #NP0323, Invitrogen) for electrophoresis, following which the proteins were transferred to a polyvinylidene fluoride membrane (PVDF) (Cat. #LC2002, Invitrogen) by using a XCell II™ Blot Module (Cat. #EI9051, Invitrogen). Note that CD9 detection was performed under non-reducing condition to reserve the epitope of the antigen52. The membrane was then blocked with 5% skim milk (Oxoid Cat. #LP0033B, Thermo Scientific) in 1×PBS Tween™ 20 buffer (Cat. #28352, Thermo Scientific) for 1 h at room temperature and incubated with primary antibodies [mouse monoclonal anti-human CD9 antibody (Cat. #10626D, Invitrogen, clone TS9, dilution 1: 750), mouse monoclonal anti-human ALB antibody (Cat. #sc-271605, Santa Cruz, clone

F-10, dilution 1:500), mouse monoclonal anti-human IgG antibody (Cat. #sc-69786, Santa Cruz, clone 3E8, 1:1000), mouse monoclonal anti-human apoA-I antibody (Cat. #sc-376818, Santa Cruz, clone B-10, dilution 1:250), mouse monoclonal anti-human apoB-100 antibody (Cat. #sc-13538, Santa Cruz, clone C1.4, dilution 1:500) overnight at 4° C. Horseradish peroxidase (HRP)-conjugated mouse IgG κ light chain binding protein (Cat. #sc-516102, Santa Cruz, dilution 1:10000) was then used to target the primaries at room temperature for 1 h. The blot was developed with SuperSignal West Femto Maximum Sensitivity Substrate (Cat. #34096, Thermo Scientific). Images were captured on a ChemiDoc MP System (Bio-Rad).

### Bottom-up Nanoflow Liquid Chromatography Tandem Mass Spectrometry Sample Preparation

Samples were lysed and digested using a modified version of the On-Micro Solid-phase Extraction Tip-based (OmSET) method, as described previously53. In brief, 20 μL of each EV isolate (concentrated from 100 μL PPP) was loaded into a 200 μL pipet tip that was packed with 2 punches of a C18 membrane (CDS Empore) from a 14-gauge blunt tip needle. Samples were lysed with 10 M urea, 2.5 M thiourea, 6 mM tris(2-carboxyethyl)phosphine (TCEP) in 30 mM ammonium bicarbonate pH 8, reduced and alkylated with 50 mM TCEP, 10 mM 2-iodoacetamide (IAA), and digested overnight with Lys-C and trypsin at 45° C. Peptides were eluted into glass LC inserts with 65% acetonitrile (ACN) in 0.1% formic acid (FA), lyophilized to dryness, and reconstituted in 5 UL 2% ACN in 0.1% FA.

### Size Exclusion Chromatography Column Optimization

An empty affinity chromatography (AC) column (Cat. #004203, Biocomma; Shenzhen, China) with a stopper was packed with 2, 3, or 4 mL of Sepharose CL-2B (CL-2B) to a bed height of 5, 6, or 7 cm, respectively. A hydrophilic filter, 8.3 mm diameter, 1.6 mm length, 50 μm pore size (Cat. #ACF-083-16-50-1, Biocomma) was placed into the column before loading CL-2B, and placed on top of the CL-2B in some column formats. The column was rinsed with two column volumes of 0.22 μm filtered 1×PBS. 100 μL of the sample (50 μL PPP mixed with 50 μL PBS) was loaded onto the column and eluted with 1×PBS. 200 μL fraction collection began (30 fractions total) once the sample was loaded. Fractions were used for BCA and TRPS analysis.

### Nano-Liquid Chromatography

An in-house bead packed C18 column was used for analysis. In short, a fused silica capillary (75 μm ID, 360 μm OD) was pulled to achieve an ESI emitter tip of ˜5-7 μm ID at the tip opening and the capillary was packed with C18 beads (Dr. Maisch, 1.9 μm, 120 Å) to 15 cm in length. An Ultimate 3000 nanoLC system (Thermo Fisher Scientific) was used for chromatography. The analytical column was connected to a piece of fused silica tubing (20 μm, 360 μm, 1 m) via a tee union, which was connected via Nanoviper to a column switching value. ESI voltage was applied to the tee union to generate nanoESI of the analytes eluting off the column. Samples were loaded at 150 nL/min for 34 min using 1% B. Analytes were eluted from the column with a flow rate of 150 nL/min using a 90 min linear gradient from 1% B to 25% B, where solvent A was 0.1% FA in water and solvent B was 0.1% FA in ACN. The solvent composition was changed from 25% B to 80% B over 2 min and held for 3 min. Lastly, the solvent composition was changed to 1% B over a period of 0.1 min and held for 15 min.

### Mass Spectrometry

Triplicate nLC-MS analyses of each sample were made using an Exploris 480 Orbitrap mass spectrometer (Thermo Fisher Scientific) coupled to an UltiMate 3000 nanoLC (Thermo). The ESI voltage was set to 1.5 kV using a Nanospray Flex Ion Source (Thermo) and the ion transfer tube temperature was set to 275° C. The instrumentation was configured in data-dependent acquisition (DDA) mode using positive polarity. Full MS1 scans were acquired from 375 to 1800 m/z at a resolution of 120,000 (at 200 m/z) with automatic gain control (AGC) target set to 3×106, maximum injection time set to 200 ms, and funnel RF level at 40. The highest abundant peaks in a cycle time of 3 s were selected for higher-energy collisional dissociation (HCD) fragmentation with charge states 2 through 5. The normalized collision energy was set to 30%. MS2 scans were collected at a resolution of 30,000 (at 200 m/z) and the isolation window was set to 2 m/z. The maximum injection time was set to 500 ms with an AGC target of 1×106, and the intensity threshold was kept at 8×103. A fixed first mass of 110 m/z. Dynamic exclusion was set to 45 s and isotope exclusion was on.

### Data Analysis

Raw files were submitted to Proteome Discoverer (v. 2.5, Thermo) using the UniProtKB/SwissProt human database (Release 2020_01, containing 20,302 sequences). Spectra were searched using the Sequest HT search algorithm with 25 ppm and 0.02 Da mass tolerance for precursor and fragment ions, respectively. Up to 2 missed cleavage sites per peptide were allowed and the minimum peptide length was set to 7 amino acid residues. Carbaminomethylation of cysteine residues was set to a static modification. Spectra were also subjected to INFERYS Rescoring with automatic mode turned on. At least one high confidence peptide was required for a protein identification at 1% false discovery rate (FDR). Label-free quantification (LFQ) was performed in Proteome Discoverer v2.5. Unique and razor peptides were used for quantification, precursor abundance was determined based on ion intensity, and p-values were calculated by the t-test. Samples were normalized based on sample volume (30 μL plasma equivalent per injection). The MS bottom-up proteomic data have been uploaded to the ProteomeXchange Consortium using the PRIDE54 partner repository with the dataset identifier (add ID number here when uploaded to PRIDE). Heat maps were generated using TBTools⁵⁵ using the Euclidean distance method. Venn Diagrams and Gene Ontology (GO) Term analysis were performed in FunRich (v3.1.3)56 using the Gene Ontology database (downloaded April 2020). The PCA plot was generated using ClustVis⁵⁷.

### Example 2

### Multimodal Stationary Phase for EV Isolation

Multimodal chromatography using CaptoCore beads was tested for binding kinetics and binding capacity properties using both protein standards and platelet-poor plasma to understand what the optimal conditions for protein depletion and EV enrichment would be. The volume of beads in suspension and the geometry of the bed packed into cartridge-format columns, as well as other operational conditions (i.e., washes, equilibration, EV isolation, incubation time) were optimized as shown in FIGS. 5A-5E and 6A-6E. The morphology of the beads was assessed by SEM imaging to understand bead diameter, shape, and pore size; CaptoCore 400 shown in FIGS. 1B-1E and CaptoCore 700 shown in FIGS. 1F-1I. Images of the beads demonstrate consistent bead and pore size distributions across all images with no visible mechanical damage or aberrations. The larger pore size of the CaptoCore 700 (FIG. 1I) media compared to CaptoCore 400 (FIG. 1E) is evident from the larger pore networks. This agrees with previously reported work that CaptoCore 400 and 700 have an average bead size of 110 μm and 91 μm with pore sizes of 19±1 nm and 50±1 nm, respectively^{36,37}. To test this, the effect of plasma treatment with CaptoCore beads alone in a tube (suspension mode) compared to plasma treatment with the CaptoCore stationary phase packed in a cartridge-type column format (without any other stationary phases) was tested to comparatively assess the performance of each CaptoCore-based mode in EV purification from plasma proteins and protein complexes. It was found that operation in suspension mode resulted in a higher binding capacity, and therefore the suspension mode was further investigated.

### Example 3

### Suspension Mode Configuration for EV Isolation

In parallel, 0.75 mL CaptoCore 400 and 0.50 mL of CaptoCore 700 were incubated in suspension mode with platelet-poor plasma. The volume ratios for the bead suspension to platelet-poor plasma were optimized prior to further experiments (FIGS. 6A-6E and 7A-7E). No leftover free plasma proteins could be detected by BCA in the supernatants of both Sus 400- and Sus 700-treated samples (FIG. 1J), indicating that the protein concentration was below the limit of detection, with the use of untreated plasma as a control. For a more thorough analysis of free plasma proteins, other analytical techniques (western blot and proteomic analysis) were employed, described below. By TRPS measurements, the EV-like particle recovery rate in Sus 400 demonstrated a recovery rate of 80.1±4% EVs from the resin, whereas Sus 700 had an EV-like particle recovery rate of 23.3±1%, with more than 70% trapped inside the pores (FIG. 1K). These recovery rates are comparable to the reported values from other commercially available size exclusion methods having ˜60-80% recovery, and ˜40% recovery for UC-based samples from plasma³⁸. The differences in EV recovery were expected, given the different MWCOs of the two resins; the larger pore size of CaptoCore 700 allowed smaller EVs and EV-like particles (e.g., exomeres and lipoproteins) into the pores, while the smaller pore size of CaptoCore 400 excluded larger proteins and protein complexes. However, smaller proteins such as serum albumin and IgGs could become trapped in the CaptoCore 400 pores, contributing to the lower levels of protein detected by BCA. To determine this effect, a more sensitive method was employed; the residual protein in each set of Sus-based EV isolation experiments was assessed by SDS-PAGE and visualized by silver stain (FIG. 8). The EV isolates prepared using CaptoCore 400 resin demonstrated a higher amount of residual plasma protein when compared to the EV-enriched samples prepared using the CaptoCore 700 resin, indicated with a red oval. This trend is also complementary to the western blot analysis (FIG. 3C). These initial results acquired using protein concentration measurements, TRPS, and SDS-PAGE indicate that, based on the difference in the pore size, the CaptoCore 400 and CaptoCore 700 stationary phases can both be used in suspension mode for EV enrichment from platelet-poor plasma, while providing a different yield and purity in EVs and EVPs.

### Binding Kinetics in Suspension Mode

Three hundred μL of CaptoCore 700 resin were washed three times with 1 mL of 0.22 μm filtered (Cat. #16534, Sartorius; Goettingen, Germany) 1×PBS by spinning at 300×g for 2 mins. The supernatant was removed after each wash. Three sets of protein standards (1:1 mixtures of IgG and BSA at 2, 6 and 10 mg of total protein. e.g., 1 mg IgG:1 mg BSA, etc.) were incubated with 0.3 mL of CaptoCore 700 resin for up to 60 min, while the supernatant was collected every 5 min for protein concentration measurements. Additionally, 1 mL of plasma sample (100 μL platelet-poor plasma, 900 L 1×PBS) was added to the washed CaptoCore 700 beads, and placed on a rotating mixer for 1 h at room temperature. A 10 μL sample was removed after every 5 min of mixing after spinning at 300×g for 2 min and used for protein quantification by BCA and separation by SDS-PAGE.

A series of time-dependent experiments were conducted to assess the binding kinetics and determine the optimal incubation time for the samples mixed with a suspension of CaptoCore 700 beads. Both protein standards and plasma samples were analyzed. To start, high-purity model protein standards, IgG and BSA, two of the most highly abundant plasma proteins, bovine serum albumin and IgG, were mixed together at a 1:1 ratio at 3 different protein amounts: 2 mg, 6 mg, and 10 mg protein. Although ˜90% of the protein was trapped inside the beads after 5 min of incubation, as determined by BCA assay, equilibrium was not reached until around 30 min of exposure at room temperature for all 3 protein amounts (FIGS. 5A and 5B). FIG. 5A shows total amount of residual protein, while FIG. 5B shows residual protein in terms of percentage. To determine if the same time was required to reach equilibrium for a complex mixture, human blood plasma at different dilution levels (from 8× to 40×) underwent the same procedures. It was found that the binding kinetics of plasma proteins were consistent with the protein standards (FIGS. 5C and 5D). FIG. 5C shows the total amount of residual protein from plasma and FIG. 5D shows the residual protein from plasma as a percentage. Plasma samples were collected at four time points (0, 5, 10, and 30 min) at four dilution levels (10×-40×) and subjected to SDS-PAGE to determine if they were also in agreement with the results of the protein concentration assays (FIG. 5E). SDS-PAGE showed a decrease in residual protein with longer incubation times across 4 different dilution levels, where the 0 min mark shows thick protein bands and the 30 min mark shows fainter and fewer protein bands. Therefore, 30 min was determined to be the optimal and sufficient incubation time. CaptoCore 400 beads demonstrated nearly identical trends (data not shown).

### Binding Capacity in Suspension Mode

0.1, 0.3, and 0.5 mL of CaptoCore 700 or 1 mL of CaptoCore 400 beads were washed as stated previously. Protein standards (IgG and BSA at a ratio of 1:1 IgG to BSA for total protein amounts of 2, 6, and 10 mg) were mixed and incubated with 0.1 mL, 0.3 mL, and 0.5 mL of the CaptoCore 700 resin, respectively. After incubation, the suspension was spun down, and the residual protein concentration in the supernatant was measured by BCA (FIGS. S2A and S2B). Additionally, 1 mL of plasma sample (100 μL PPP, 900 μL 1×PBS) was added to each, and tubes were placed on a rotating mixer. After 30 mins, samples were spun at 300×g for 2 mins, and the supernatant was removed for BCA and SDS-PAGE.

As in the binding kinetic experiments, protein standards and human blood plasma were used to determine the binding capacity of the CaptoCore 700 resin in suspension mode. Volumes of resin ranging from 0.1 mL-0.5 mL resin (FIGS. 6A-6B) were tested, and it was found that higher efficiency of protein depletion was obtained with higher resin volumes. FIG. 6A shows the total amount of residual protein for 3 different volumes of resin. FIG. 6B shows residual protein in terms of percentage, with 0% protein detected using 0.5 mL resin for each protein amount. From these data, using Equation 1, the maximum binding capacity of CaptoCore 700 was determined to be 20.1±0.5 mg protein/mL CaptoCore 700 resin:

$$Binding capacity = \frac{initial protein amount - residual protein amount}{volume of stationary phase} \qquad ( 1 )$$

Similar experiments were performed with plasma at a series of dilution levels (from 5× to 20×) (FIGS. 6C-6D). FIG. 6C demonstrates a similar trend that the amount of residual plasma protein detected decreases as the volume of resin used increases. The rate of depletion was similar among all dilution levels (i.e.). FIG. 6D illustrates depletion in terms of percentage of residual protein detected. Both FIGS. 6C and 6D show total detectable protein depletion at most dilution levels by using 0.5 mL resin, indicating CaptoCore is suitable for the use of not only protein standards, but for plasma as well. Using Equation 1, the determined maximum binding capacity using plasma was 23.75±0.5 mg of free plasma protein/mL CaptoCore 700 resin. Next, SDS-PAGE was performed, which also confirmed that residual protein in solution decreased when plasma samples were incubated with larger volumes of CaptoCore 700 (FIG. 6E). The similarity between the determined values in the two evaluated sample systems indicates that the CaptoCore 700 stationary phase demonstrates comparable protein binding capacities for both pure protein standards and complex plasma samples. Similar experiments were performed with CaptoCore 400 in suspension mode, resulting in a higher EV recovery rate (FIG. 1K), where 80.1±1% EVs were recovered from plasma.

Optimized EV Isolation by Suspension Mode

One hundred μL PPP was diluted by 10× with 1×PBS before incubating with either 0.75 mL CaptoCore 400 or 0.5 mL CaptoCore 700 resin on a mix-rotator at room temperature for 30 min. The mixture was then spun down at 300×g for 2 min, and the supernatant was carefully collected. The supernatant was concentrated down to ˜30 μL using a centrifugal filter (MWCO 30 kDa, Amicon).

### Example 4

### Size Exclusion Chromatography (SEC)-Based EV Isolation

Different column lengths of SEPHAROSE CL-2B were tested for SEC-based EV enrichment from platelet-poor plasma as a baseline for a regularly used SEC method. Columns were packed in-house with 2 mL, 3 mL, or 4 mL of CL-2B stationary phase, corresponding to bed lengths of 3.5 cm, 5.25 cm, or 7 cm, respectively. 100 μL of plasma was loaded onto each column, and 200 μL fractions of eluent were collected and initially characterized by SDS-PAGE, TRPS, and BCA (FIGS. 15A-15D). The combination of TRPS assessment and BCA protein concentration measurements allowed determination of the fractions in which either EVs or free plasma proteins and protein complexes were eluting. The resolution between the chromatographic peaks corresponding to EVs and free plasma proteins improved as the bed volume increased. EVs and EV-like particles were mainly enriched in fractions 4-6 using the 2 mL column (FIG. 15A) and in fractions 6-8 using both the 3 mL (FIG. 15B) and 4 mL (FIG. 15C) columns. The majority of free plasma proteins were detected in fractions 6-13 in the 2 mL column (FIG. 15A), causing significant overlap with the EVs. The protein peak shifted to fractions 9-21 in the 3 mL column (FIG. 15B), still causing some overlap with the EVs. Finally, minimal overlap was observed in the 4 mL column (FIG. 15C), where proteins eluted in fractions 10-23, with minimal overlap. It was determined that the bed volume of 4 mL and column length of 7 cm provided sufficient resolution between the EV and free plasma protein peaks for the selected plasma volume of 100 μL (after 2-fold dilution; FIG. 15C). Therefore, the 4 mL SEC column bed was used in subsequent experiments, labeled as “SEC” in other sections. Also, it was assessed whether the addition of a hydrophilic filter as a retaining frit on the top of the column could improve the performance of EV isolation (FIG. 15D). The filter reduced the disruption of the packed resin during sample loading. However, adding the filter did not noticeably alter the EV isolation performance, and the column preparation required an additional step. Therefore, it was decided not to use a filter as a retaining frit at the inlet of the column, but to keep a frit on the outlet of the column.

### Example 5

### Sandwich Column Configuration for Multimode Chromatography-Based EV Isolation

The separation efficiency of the sandwich column format for both CaptoCore 400 (SW 400) and CaptoCore 700 (SW 700) fractions was assessed in the same way as for the SEC column fractions using TRPS, BCA assay, and silver-stained SDS-PAGE (FIGS. 9A-9F). For the initial optimization experiments, 3 mL or 4 mL Sepharose CL-2B was packed into a column and then topped by a layer of either 0.75 mL CaptoCore 400 (FIG. 9A-9B) or 0.5 mL CaptoCore 700 (FIG. 9C-9D) resin. Platelet-poor plasma was loaded onto the SW columns with the outlet closed with a stopper to allow for interaction of the loaded sample with the layer of the CaptoCore beads. Based on the binding kinetic results shown in FIGS. 5A, 5C, 10 min incubation was used before elution and fraction collection for plasma. EVs were mainly enriched in fractions 7-9 in the SW 400 column (FIGS. 9A, 9B), while in the SW 700 column, the EV peak was mainly in fractions 6-8 (FIGS. 9C, 9D). Interestingly, the protein peaks were lower in using CaptoCore 400, possibly due to CaptoCore 700 trapping more lipoprotein particles, discussed below by the western blot and proteomic data, allowing for more free plasma proteins to be detected by BCA. When comparing the sandwich columns to a standard CL-2B column (FIG. 1L-N) the protein peaks in the sandwich columns resulted in lower peak intensity and smaller area of the protein peak, indicting higher rates of protein depletion. The addition of a CaptoCore layer also resulted in a higher resolution between the EV and protein peaks and, therefore, in an increased purity of EV isolates compared to the EV enrichment using Sepharose CL-2B alone. The efficient purification of the EV-rich fractions from free plasma protein was further confirmed for both SW 400 and SW 700 columns by SDS-PAGE (FIG. 9E-9F).

Assessment of Binding Capacity in Column Format. CaptoCore 700 binding performance was investigated in a column format alone without additional stationary phases. 0.5 mL, 0.75 mL, or 1 mL of the CaptoCore 700 resin was packed into a cartridge-type column, which was operated under gravity-flow mode (FIGS. 16A-16D). A total volume of 100 μL of protein standard (IgG: BSA, 1:24 (w/w); total protein amounts range from 2.5 mg to 25 mg) was loaded, fractions were collected, and residual protein amount was measured by BCA. The same protocol was carried out with 100 μL of human blood plasma at different dilution levels to keep the total amounts of plasma proteins at 2.65 mg, 5.6 mg, and 10.6 mg (FIGS. 16C-16D). While the packed CaptoCore stationary phase also resulted in efficient protein depletion, the substantially reduced interaction time between the sample and the resin in the column format did not allow the maximum binding capacity to be achieved compared to suspension mode.

### Sandwich Column Mode Optimization

An empty AC column (Biocomma; Shenzhen, China) was packed with Sepharose CL-2B resin with either 0.75 mL of CaptoCore 400 or 0.5 mL of CaptoCore 700 on top of the Sepharose CL-2B. A filter (Biocomma; Shenzhen, China) was placed between the two resin layers in some column formats. The column was rinsed with two column volumes of 0.22 μm filtered 1×PBS. After rinsing, a stopper was placed onto the column outlet, and 100 μL of the sample (50 μL platelet-poor plasma (PPP) mixed with 50 μL 1×PBS) was loaded on the top of the column. The CaptoCore 400 or CaptoCore 700 beads were, allowed to incubate for 10 mins, the stopper was opened, and fractions were collected as in the same manner as the SEC column optimization.

### Optimized EV Isolation by Sandwich Mode

One hundred μL PPP was diluted by 1.5× with 1× PBS and then loaded onto a SW column with 7 cm CL-2B and an additional 0.75 mL CaptoCore 400 or 0.5 mL CaptoCore 700 resin on the top. Elution started after 10 min of incubation was allowed. After the void volume, the first four fractions of 200 μL each were collected by an Automatic Fraction Collector (AFC, iZon Science Ltd.; Christchurch, New Zealand) and then pooled together before being concentrated to ˜30 μL using a centrifugal filter (MWCO 30 kDa, Amicon).

### Example 6

### Assessment of EV Isolation Methods by Intact EV and Protein Analysis

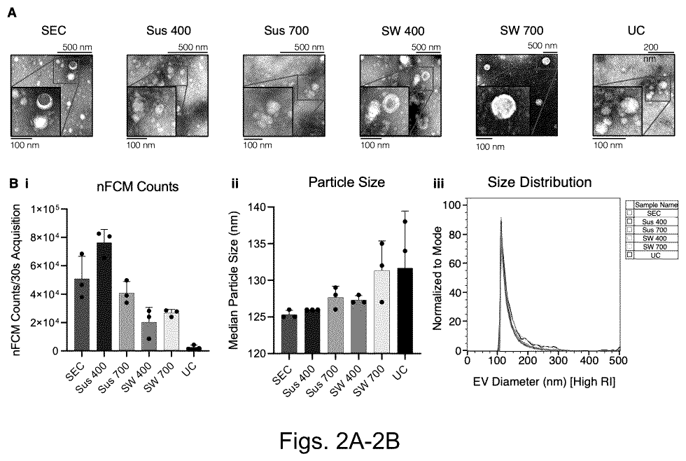

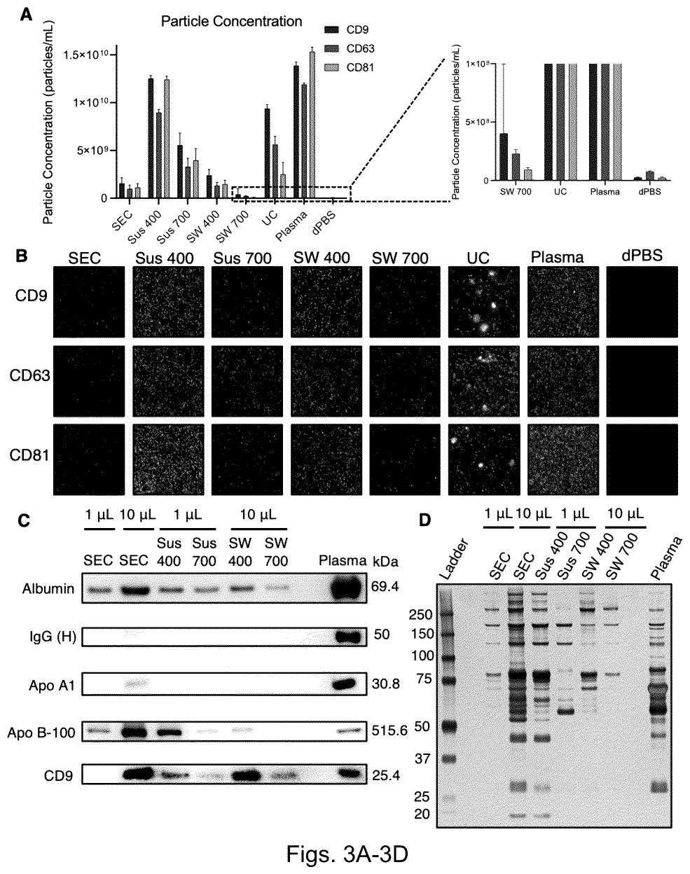

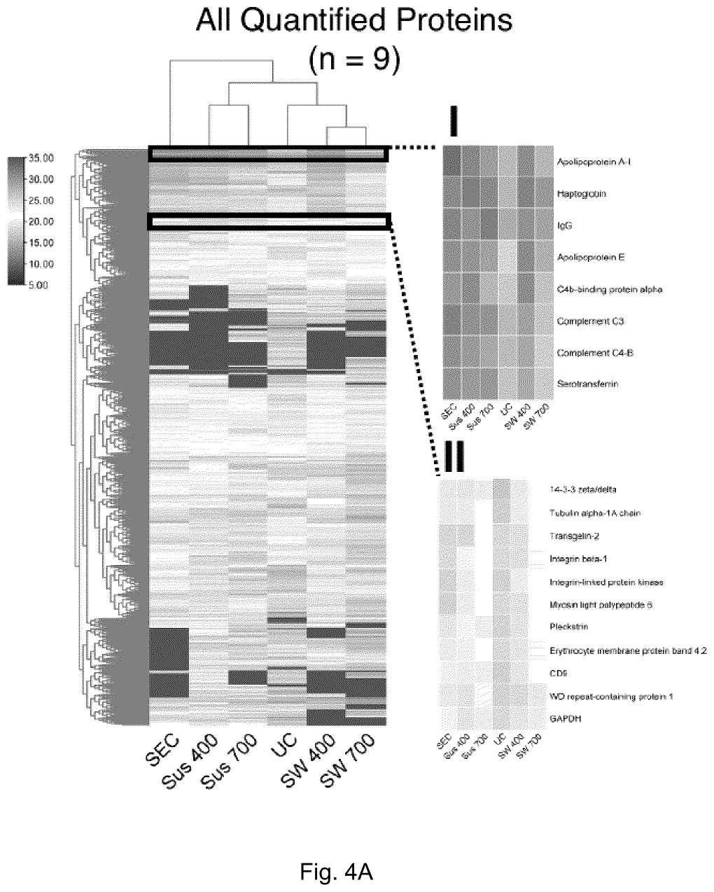

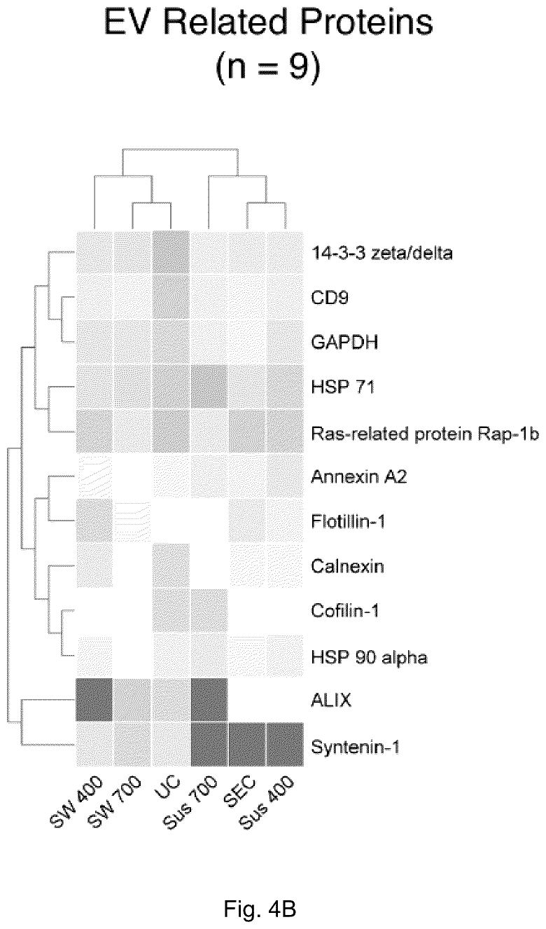

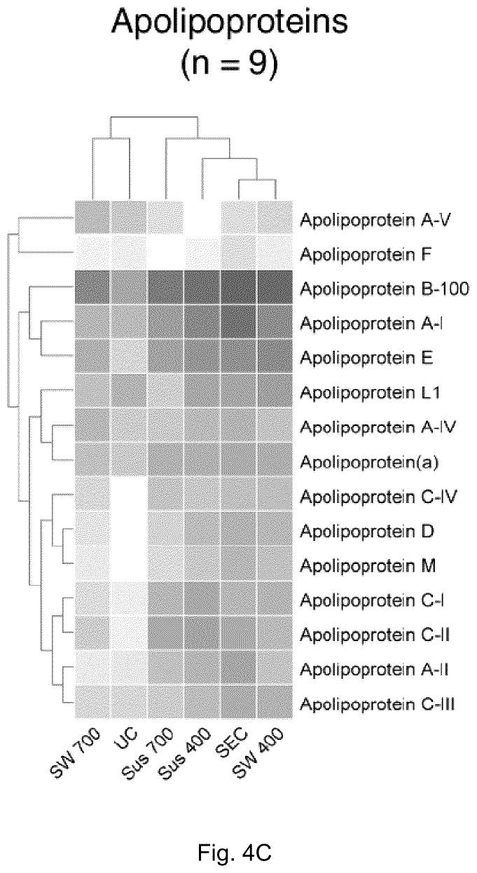

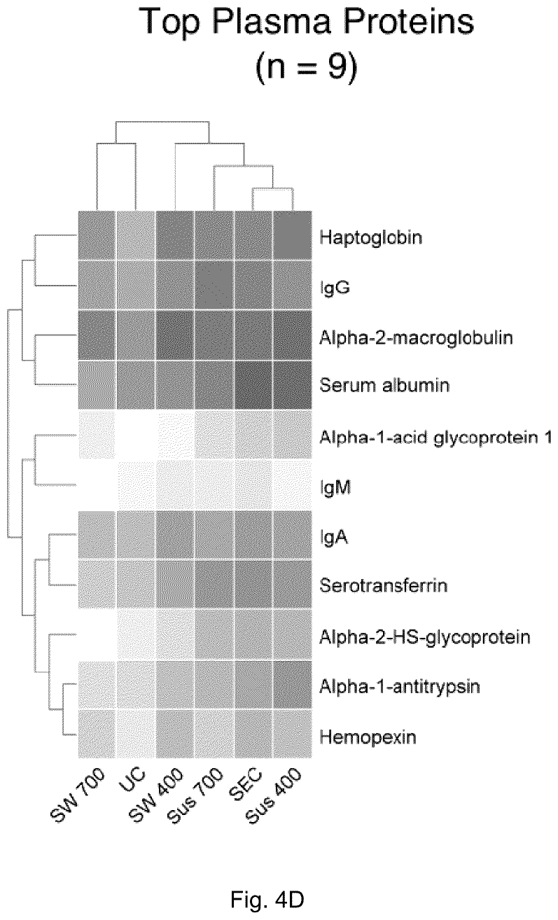

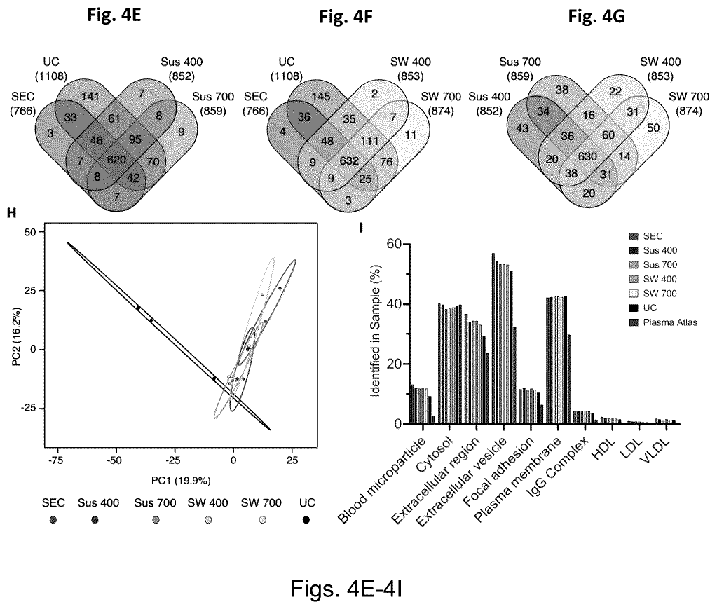

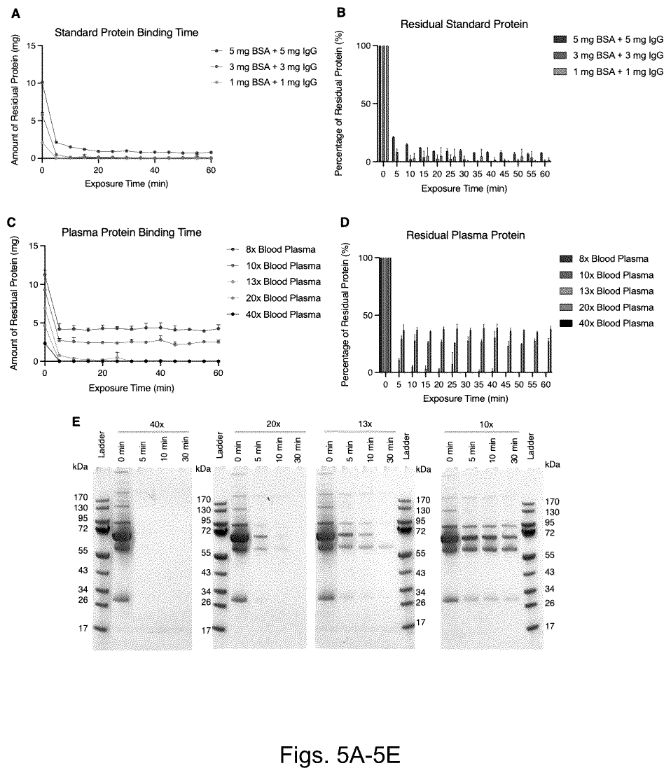

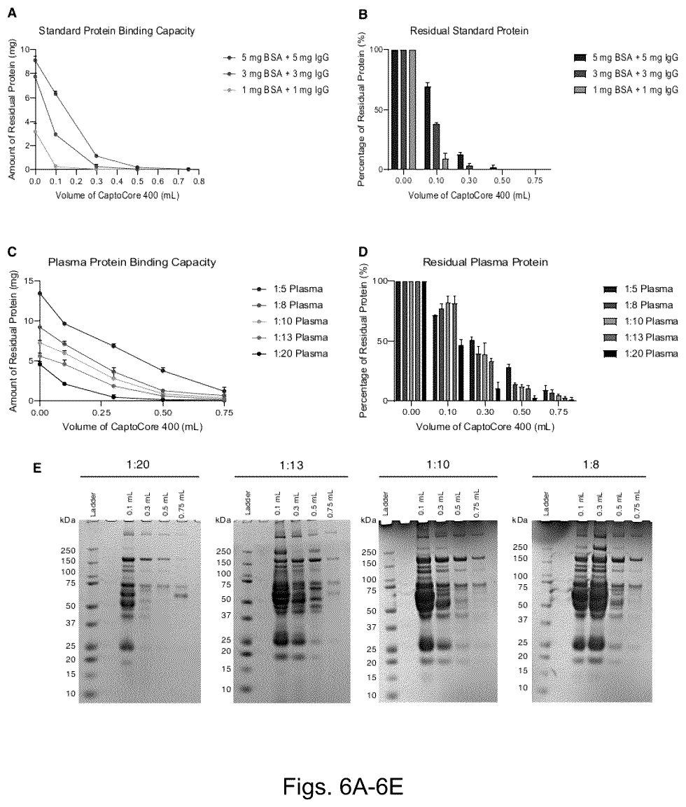

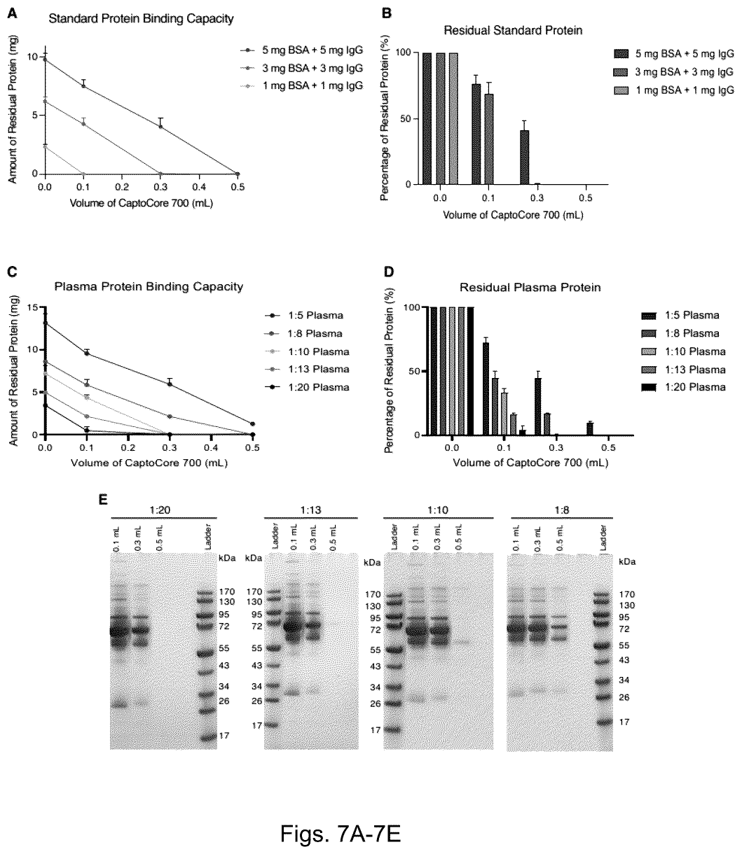

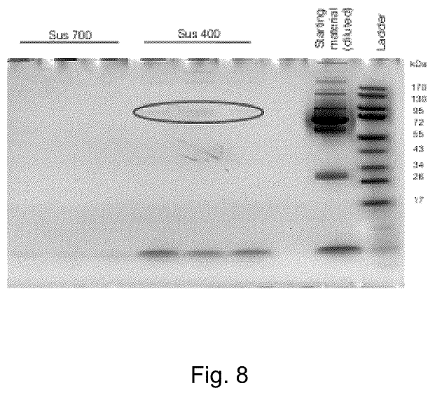

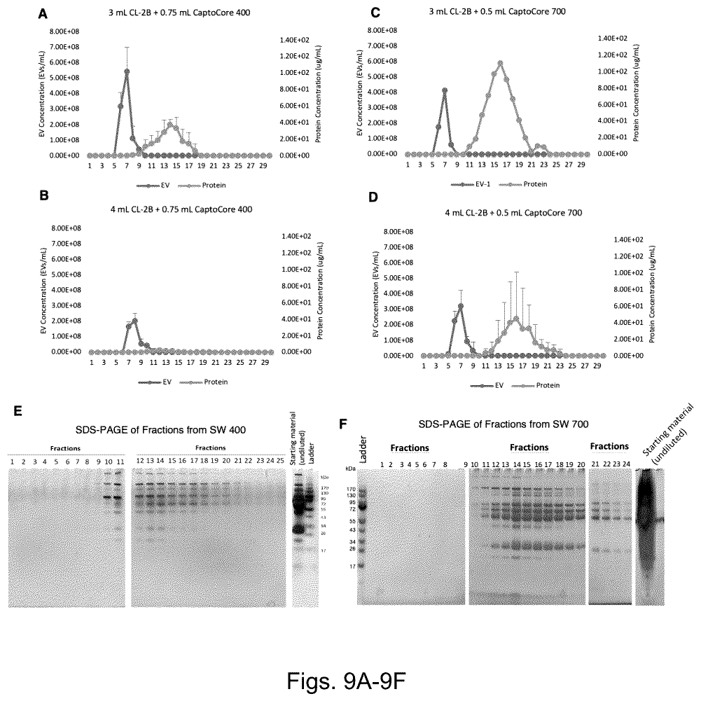

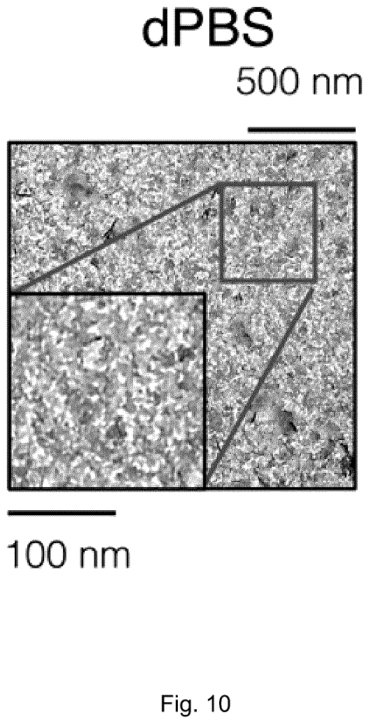

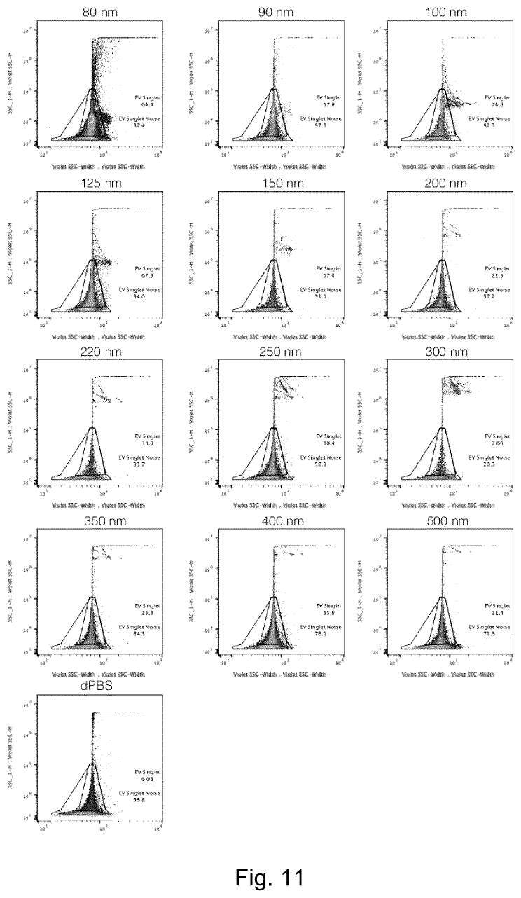

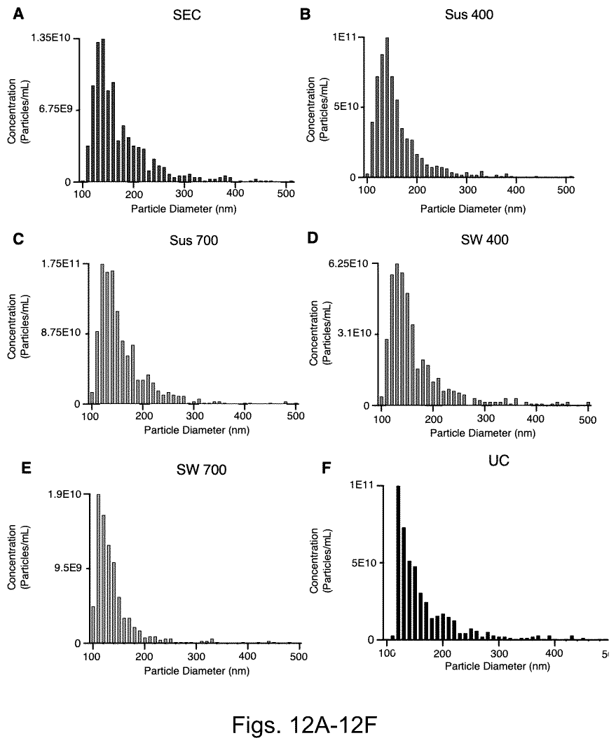

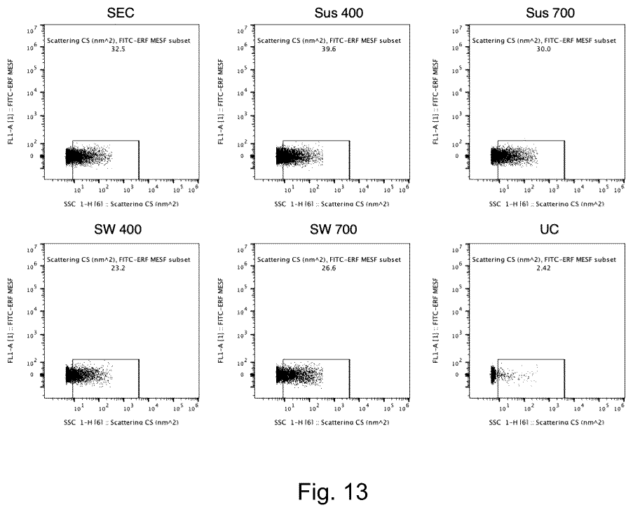

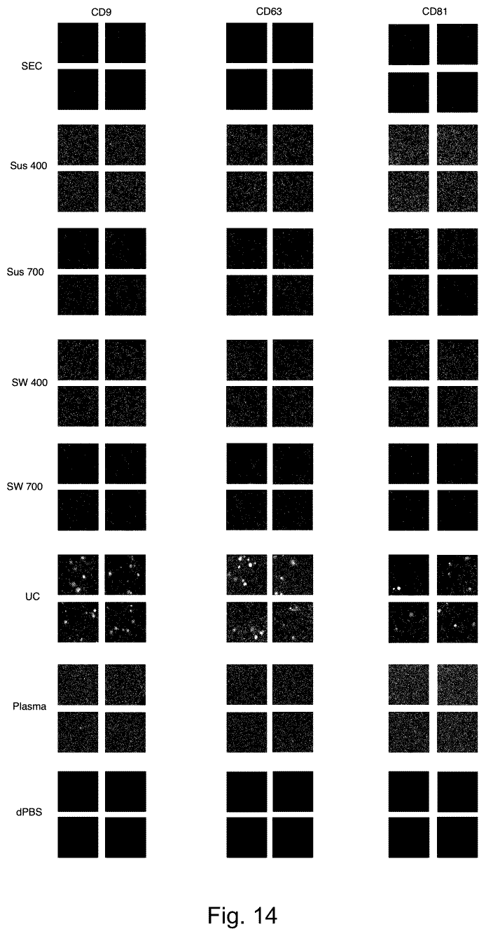

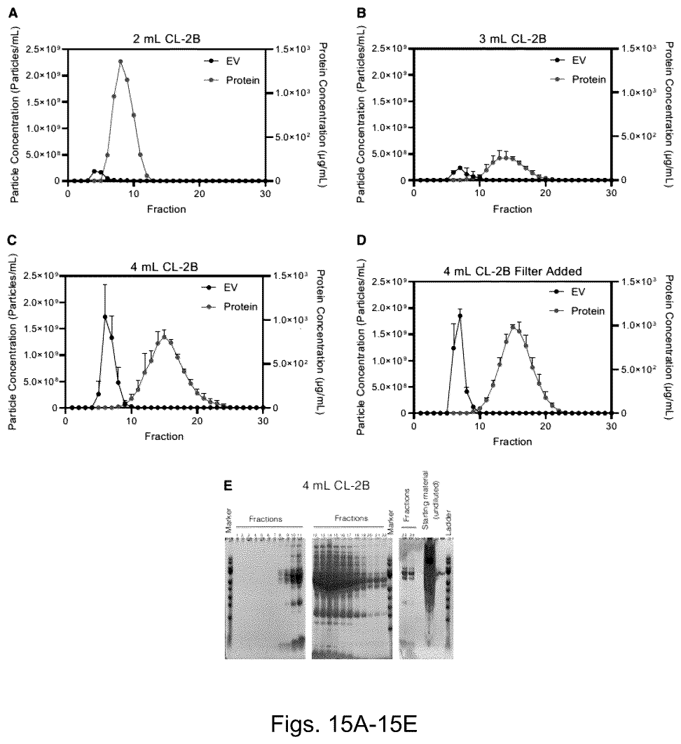

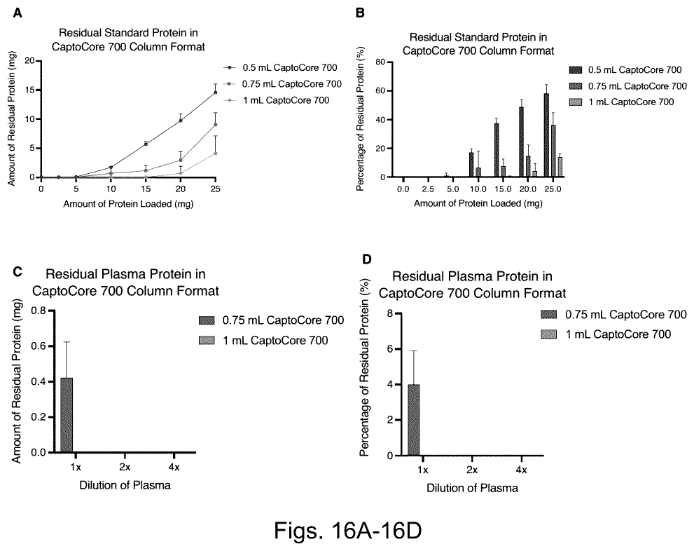

First, TEM imaging was used to visually confirm the presence of EVs in each of the EV isolates as well as to compare the size and morphology of EVs isolated by each of the evaluated techniques (FIG. 2A). Through TEM analysis, all examined techniques resulted in successful EV isolation, which was evident from the detection of classically shaped EV-like structures of similar size and morphology characteristic to EVs. TEM confirmed that the EVs that were isolated exhibit visually similar morphology characteristics such as cupping, a common EV characteristic when samples are dried^{39,40} Based on the TEM imaging, the EV size distributions were similar for the different evaluated isolation techniques, which can be seen by the inset images and by TRPS data (FIGS. 2A and 11). As a negative control, dPBS was assessed, with no EV-like features detected (FIG. 10).

Furthermore, the performance of EV enrichment was characterized using side scatter measurements from nanoflow cytometry (FIG. 2B). Samples were gated based on a negative control, dPBS, and calibrated polystyrene beads (PS beads) ranging from 80-500 nm in diameter (FIG. 11), ensuring noise detected from the dPBS was not selected within the gate. Particle counts per acquisition time correlate to the approximate concentration of EVPs and other nanoparticles within the samples (FIG. 2Bi). An expected trend was observed, where Sus 400 had the highest counts out of the CaptoCore-based samples, followed by Sus 700, and the sandwich columns. Interestingly, the SW 400 and SW 700 columns were similar in their counts per acquisition time, within error. Median particle diameter was obtained first by measuring the side scatter and refractive index (RI) of each PS bead and extrapolating to the measured side scatter and RI of each EV isolate, using methods shown in previous work⁴¹⁻⁴³ (FIG. 2Bii). The measured size distributions show that most of the isolated EVs were <200 nm in diameter, which was also observed with TRPS measurements (FIG. 11). On average, SEC showed the lowest median size distribution with 125.3±0.6 nm measured between replicates. The use of CaptoCore in suspension mode shifts the population slightly higher, with the median size of Sus 400 and Sus 700 measured to be 126.0±0.0 nm and 127.7±1.5 nm, respectively. Using CaptoCore in sandwich mode shifts the population higher as well, with the median size of SW 400 and SW 700 measured to be 127.3±0.6 nm and 131.3±4.0 nm, respectively. The shift in size distributions can be attributed to smaller EVs, apolipoproteins, and other nanoparticles becoming trapped inside the internal positively charged surface of the CaptoCore beads, leading to the detection of larger particles. CaptoCore 700 has an even larger pore size, letting in more particles to be trapped inside the pores, compared to CaptoCore 400, shifting their median sizes higher in both cases of suspension mode and sandwich mode. However, the UC-derived isolate showed lower EV signal (counts per acquisition) with larger particle sizes on average 131.7±7.8 nm, presumably from obtaining larger EVs and EV aggregates during the harsh isolation process, which agrees with previously reported size distributions post-UC processing²⁵. However, an overlaid histogram of all isolation methods shows the overall similarity of each technique, each isolating EVs in the exosome size range (FIG. 2Biii). By comparing the nFCM to TRPS size histograms (FIG. 11), they are similar and complementary to each other, with the majority of particles ranging from about 100 to about 200 nm in diameter, indicating that CaptoCore is suitable for EV enrichment. To ensure nFCM instrument consistency between runs, scattering cross sections for each isolation method were also assessed, ranging from 2.42 to 39.6 nm² with the noise kept constant and not included within the gate.

### Example 7

### Molecular Characterization of Isolated EVs

Immunoaffinity-based interferometry is a light-based analytical technique that has been shown to be useful for nanoparticle analysis, based on a single particle interferometric reflectance imaging sensor to count and image fluorescently stained EVs on a chip⁴⁴. This technique was employed to qualitatively and quantitively assess EV enrichment and to comparatively phenotype EV isolates based on EV-characteristic protein markers. Quantitively, EV isolates generated using the developed and conventional EV purification approaches were fluorescently tagged and counted for three commonly probed EV protein membrane markers: CD9, CD63, and CD81 (FIG. 3A).

Each isolation method showed the detection of EVs, with a clear pattern emerging. The use of both CaptoCore 400 and 700 in suspension mode increased EV recovery compared to a CL-2B SEC column, which was complementary to intact EV nFCM data, within error (FIG. 2Bi). These immunoaffinity-based interferometry data showed Sus 400 having the greatest yield with 82.5% EV-like particles recovered followed by 30.2% EV-like particles recovered in Sus 700 with the SW 400 column, CL-2B column, and SW 700 column following. Similarly, the Sus-based recovery rates obtained by TRPS measurements (FIG. 1K) indicated the same trend of Sus 400 having a higher yield than Sus 700. The use of SW columns decreased the EV recovery rate, but upon further analysis, discussed in more detail below, increases the purity of the EV isolation relative to Sus-based samples. Platelet-poor plasma was used as a positive control and dPBS was used as a negative control. Qualitatively, interferometry was used to image the samples based on the detection of the above markers (FIG. 3B). Three fluorescent channels were used for the three EV proteins of interest: CD9—blue, CD63—red, CD81—green. Due to the possibility that an EV can have more than 1 of these proteins on its surface, multiple tags can be observed in each channel (i.e., red and green tags can be seen in the blue CD9 channel). Interestingly, the gold standard technique for EV isolation based on UC appeared to consistently result in aggregation and clustering of EVs, and possibly proteins and lipoprotein particles, shown as large purple clumps. These images agree with previously reported data that conventional UC-based isolation techniques can cause EV loss and lipoprotein-EV combined aggregation with each centrifugation step⁴⁵. In contrast, all other evaluated multimode chromatography-based EV isolation techniques did not show any signs of EV fusion or protein aggregation based on the immuno-interferometry assays. Replicate images are shown in FIG. 14.

The EV isolates were assessed by western blotting against CD9 and the highly abundant plasma proteins, serum albumin, IgG, apolipoprotein A1, and apolipoprotein B100, that are known to co-isolate with EVs, especially by UC³ (FIG. 3C). The western blotting showed successful depletion of these highly abundant plasma proteins across all isolation methods, relative to the starting platelet-poor plasma samples. One μL plasma equivalent for Sus-based samples was used as to not overwhelm the signal and 10 μL plasma equivalent for SW-based samples was used to have enough detectable signal.

For comparison, 1 μL and 10 μL plasma equivalents of a SEC-based sample was used in the first 2 lanes of the western blot, respectively. Noticeably, a trend appears where all protein bands appear weaker in both CaptoCore 700-based samples compared to CaptoCore 400 samples for each respective isolation mode. However, when comparing the 1 μL Sus-based samples to 1 μL SEC sample, the CD9 band is more intense with similar intensities of plasma proteins which indicates a higher concentration of EVs in Sus-based samples than in SEC, complimenting nFCM and TRPS data. Comparing the 10 μL SW-based samples to the 10 μL SEC sample, the plasma proteins appear weaker in the SW-based samples with CD9 to have a comparable intensity, with slight loss in the SW 700 sample. This indicates lower free plasma protein contamination with similar EV concentrations. These trends can also be observed by SDS-PAGE, where the total protein content is lower in CaptoCore 700 samples relative to their CaptoCore 400 counterparts (FIG. 3D). Moreover, similar to the the western blot, the CaptoCore-based methods show better free plasma protein depletion efficiency compared to their equivalent volumes of the SEC-based EV isolation method.

### Example 8

### Proteomic Analysis of Isolated EVs

NanoLC-MS/MS-based bottom-up proteomic profiling was performed across all samples to identify and quantify proteins recovered by each EV isolation method. The abundance levels of the most predominant plasma proteins (serum albumin, lgG, etc.) 46 and selected EV-characteristic proteins (CD9, annexin A2, etc.) using guidelines from MISEV2018 and other reports^{3,7,47,48} were compared for their relative abundances (FIGS. 4A-4D). Unsupervised hierarchical clustering conducted on the quantitative proteomic data derived from the different modes of EV isolation demonstrated a substantial similarity between all generated proteomic EV profiles in general, where SW-based samples cluster together and show a significant degree of similarity with the UC-based isolate (FIG. 4A). Sus-based samples also cluster together and the SEC-based isolate showed the highest degree of difference from the rest of the samples. In general, the modes of isolation perform similar to each other, where there are slight differences in isolation of EVs and protein depletion depending on the resin and mode of isolation used. Zooming into the topmost clusters, the more abundant plasma proteins (apolipoprotein A-1, haptoglobin, IgG, etc.) are reliably quantified without causing major interference with the detection and quantitation of lower abundance proteins (FIG. 4Ai). The UC- and SW 700-based techniques resulted in the higher depletion efficiency for the highest abundance free plasma proteins according to the measured protein abundance levels. After sample processing by the assessed EV isolation techniques, the dynamic range of plasma and

EV proteins abundance levels decreases, allowing lower abundance EV proteins to be reliably detected and quantitated in another cluster (FIG. 4Aii). EV-related proteins such as 14-3-3 zeta/delta, CD9, and GAPDH naturally cluster together, indicating similarities among these proteins and, therefore, similarities among the EVs when they are isolated, as opposed to randomly being dispersed within the hierarchical clustering.

Several EV-related proteins were comparatively examined using the same hierarchical clustering approach based on their measured abundances (FIG. 4B). Overall, all evaluated EV isolation techniques demonstrated largely similar patterns in protein abundances. However, each method showed specific differences in abundance of the EV-related proteins of interest. Specifically, the SEC and Sus 400 samples showed the highest correlation in clustering, closely followed by SW 700 and UC preparations thereafter. Sus 700 and SW 400 clustered further away, presumably due to their low recovery of ALIX, a cytosolic protein involved in multivesicular body formation through the endosomal sorting complex required for transport (ESCRT) pathway⁴⁹. Additionally, each isolation method does not recover all EV-related proteins equally. For example: UC-based isolation has a ˜10-fold decrease in abundance in annexin A2, a membrane-binding protein involved in Ca²⁺ regulation, compared to Sus 400. While UC did recover 14-3-3 zeta/delta, CD9, and GAPDH at higher abundances compared to other methods, the CaptoCore-based methods demonstrated the ability to also recover these proteins. ALIX, flotillin-1, cofilin-1, and syntenin-1 exhibited the most different trends from other EV-characteristic proteins between the tested methods, which may indicate that different EV isolation techniques have certain preferences and biases in the recovery of the specific EV and EVP types depending on where the proteins are localized within the cell at the time of EV formation. Overall, the UC-, SEC-, and Sus 400-based isolation methods showed slightly higher recoveries of EV-related proteins compared to the others, which agrees with our TRPS and nFCM data. By assessing the quantitative profiles of apolipoproteins (FIG. 4C), different recovery patterns were observed than EV-related proteins. The SEC, Sus 400, and SW 400 methods demonstrated the lowest efficiency in lipoprotein depletion, on average, and particularly, for apolipoproteins A-1 and B-100. The SW 700 column- and UC-based techniques showed the highest depletion efficiencies for apolipoproteins, specifically this is exemplified for the apolipoproteins with lower molecular mass (i.e., apolipoproteins A-II, C-I, M, etc.) where more of these particles can fit inside the CaptoCore 700 pores. Furthermore, SW 700 has, on average, ˜60-fold decrease in abundance in apolipoprotein A-1 compared to the standard CL-2B SEC column and overall ˜16-fold lower levels of all apolipoproteins compared to CL-2B SEC column. This indicates higher depletion efficiency for apolipoproteins with the combined use of CL-2B SEC and CaptoCore 700 resin. Similarly, the recovery rates based on abundance levels of the representative most abundant plasma proteins was examined as well (FIG. 4D). Overall, similar to the trend observed for lipoprotein depletion, the UC and SW 700 methods demonstrated the significantly higher depletion efficiency for top plasma proteins (e.g., serum albumin, IgG, IgA, haptoglobin, serotransferrin, and alpha-1-antitrypsin) compared to the standard SEC CL-2B column and other CaptoCore-based methods. Serotransferrin, for example, showed a 25-fold decrease in abundance in the SW 700 column compared to the SEC-based approach. Again, this can be attributed to the combined action of the multimode bilayer SW 700 columns, where the free plasma proteins and protein complexes are becoming efficiently trapped inside the CaptoCore beads and the remaining species are better separated in the SEC dimension during processing because of the lower dynamic range of the non-EV species. It was also observed that the use of CaptoCore alone (both Sus 400 and Sus 700) does not deplete apolipoproteins and free plasma proteins as well as in a column format (i.e., Sus 400 has ˜10-fold higher serum albumin compared to its SW 400 counterpart and Sus 700 has ˜7-fold higher serum albumin compared to SW 700). This trend is observed in almost all free plasma proteins as well as in apolipoproteins, where the use of CaptoCore in a column format has higher depletion rates than in suspension mode. These data reflect the difference in the pore size of CaptoCore beads and complement our hypothesis that the CaptoCore 700 depletes more free plasma proteins (e.g., serum albumin, IgG, serotransferrin) and highly abundant lipoprotein particles of various types compared to the other evaluated CaptoCore-based formats. Specifically, the SW 700 column also enriches EVs, based on abundance levels of EV-associated proteins (e.g., CD9, annexin A2, 14-3-3 zeta/delta) to a similar efficiency to UC-based samples and at a higher efficiency to standard SEC columns.

Next, the similarities and differences in the total number of proteins was quantified for different isolation methods using Venn diagrams (FIGS. 4E-4G). While many of the quantified proteins were common to the different isolation methods, their relative abundances were shown to be different, as discussed above (see also FIGS. 4A-4D). Comparing conventional EV-isolation techniques to the Sus-generated samples, the majority of the proteins are shared between these methods (FIG. 4E). Noticeably, 141 proteins are unique to UC, many of which are not related to EVs, and some are plasma-associated proteins (e.g., IgGs). A similar outcome occurs when comparing the conventional techniques to the SW-based EV isolation methods; the UC-generated isolates resulted in the detection of 145 unique proteins, many of which are not EV-related (FIG. 4F). In both FIGS. 4E and 4F, the CaptoCore samples have a small number of proteins that are unique and specific to them, indicating a significant similarity of the compared EV isolation techniques based on the proteomic profiles generated using both conventional (SEC and UC) and CaptoCore-based techniques. There also appears to be a significant overlap in profiled proteins when comparing all CaptoCore-based EV isolation techniques, with the majority of the proteins shared between methods (FIG. 4G). Overall, taking into account the number of proteins identified by each method, what proteins they are, and their abundances, our comparative analysis indicates that there is a similar efficiency of plasma protein depletion and EV purity in the UC- and SEC-generated samples compared to CaptoCore-based EV isolates. Furthermore, a principal component analysis (PCA) plot highlights the differences and similarities between the evaluated EV isolation techniques based on the results of qualitative and quantitative proteomic profiling of the EV isolates (FIG. 4H). While all CaptoCore isolates exhibit similarities by clustering together, UC-derived isolates resulted in a distant cluster shifted away from the rest based on both PC1 and PC2, indicating the types of proteins and protein amounts that are being isolated by the UC-based approach are significantly different from the isolates purified using the SEC- or CaptoCore-based methods.

Gene Ontology (GO) term enrichment analysis (FIG. 4I) shows the percent of enrichment for several cellular components of interest compared to the total proteins identified. Every isolation method yields the highest percentage of proteins that are associated with EVs, followed by the plasma membrane, cytosol, and extracellular region. Many proteins associated with EVs originate from the cytosol or the plasma membrane (e.g., CD9 is a membrane protein as well as an EV-related protein) and will contribute to both GO Terms. As a reference, all isolation methods were compared to the Human Plasma PeptideAtlas⁵⁰ (built July, 2021) which contains ˜4,400 canonical proteins. The use of CaptoCore yields in similar percentages of EV-related proteins compared to the SEC- and UC-generated samples, indicating the presently developed methods are optimized for EV isolation. Plasma protein terms (lgG complex, HDL, LDL, VLDL) were included to illustrate the percentage of plasma proteins identified in each sample is minimal (<10%).

### Example 9

### Comparison of Suspension Mode and Column Format for EV Isolation

Using two types of CaptoCore resins in two different modes, alone in suspension (Sus) or packed in a multiphasic column format (SW), parameters and procedures were optimized for the isolation of EVs from plasma. The present multimode chromatography-based techniques offer cost-efficiency, scalability, and higher throughput for EV isolation compared to UC-based approaches. This advantage is especially pertinent for potential clinical applications, where UC-based EV isolation is low throughput, requires expensive equipment, and can cause aggregation of EVs, proteins, and lipoprotein particles during the isolation process. Moreover, the use of CaptoCore resin instead of a traditional CL-2B SEC column can offer higher or similar EV recovery rates, depending on the mode of separation, measured by TRPS and interferometry, with higher free plasma protein depletion, assessed by the western blot and proteomic data.

To understand how CaptoCore resin would interact with EVs and plasma proteins in Sus mode, the binding kinetics and binding capacity of the resin were tested (FIGS. 5A-5E and 6A-6E) respectively, showing equilibrium was reached after 30 mins of incubation and that the maximum binding capacity was determined to be 23.75±0.5 mg plasma protein per 1 mL CaptoCore resin. The resins were also tested to determine depletion of free protein amount by BCA and intact EV recovery rate by TRPS (FIGS. 1J and 1K) respectively. This showed no detection of plasma protein in both resins and comparable EV recovery amounts to previous reports. SEM imaging was employed to inspect the CaptoCore resin and confirmed the beads are nearly spherical in shape, with no visible abreactions (FIGS. 1B-1I). The relative pore size difference between CaptoCore 400 and CaptoCore 700 was also confirmed, with the 700 media having larger pores. Furthermore, TEM imagining was used to visually confirm the presence of EVs by the different isolation types (FIG. 2A). Once CaptoCore was determined to be suitable for EV enrichment, further, intact particle analysis was performed by nFCM and immunoaffinity-based interferometry (FIGS. 2B and 3A-3B), respectively. Using nFCM, median particle size diameters were obtained, showing that each tested technique isolates particles with similar diameters. It should be noted that the limit of detection is ˜100 nm for a biological structure in both analysis types of nFCM and TRPS, which also hinders the detection of smaller EVs. Immunoaffinity-based interferometry demonstrated intact EV particle recovery, with both Sus-based methods outperforming a conventional CL-2B SEC column, and Sus 400 recovering more particles than UC-based isolation. The SW-based columns showed a recovery rate similar to a conventional CL-2B SEC column. Furthermore, fluorescence images revealed evidence of particle aggerates during UC isolation with all over techniques showing distinct individual particles. Obtaining individual particles is needed for several applications where it is critical to preserve EV structure and morphology.

Protein recovery was assessed by the western blot, SDS-PAGE, and mass spectrometry. All tested isolation methods indicated depletion by the western blot against 4 of the top most abundant plasma proteins compared to plasma without EV enrichment: serum albumin, IgG (H), apolipoprotein A1, and apolipoprotein B-100 (FIG. 3C). CD9 was used to probe for EV recovery, with enrichment in all developed techniques, with CaptoCore 400-based samples yielding higher recoveries compared to their CaptoCore 700 counterparts. SDS-PAGE compliments this as well, with total protein amount to be higher in CaptoCore 400 samples compared to CaptoCore 700-based methods (FIG. 3D). The mass spectrometry proteomic profiling experiments demonstrated that the abundance levels of all proteins in the EV isolates generated using the SEC CL-2B- and UC-based techniques were similar to that of the developed multimode chromatography techniques, indicating similar EV purification efficiencies, with slight differences (FIG. 4A). Abundance levels were also assessed for select EV-related proteins, apolipoproteins, and top plasma proteins with CaptoCore 400 samples having slightly higher protein abundances compared to CaptoCore 700 samples (FIGS. 4B-4D). Upon further analysis, Venn Diagrams confirmed that the developed methods are comparable to each other in the number of proteins quantified, having the majority of the proteins (<600 proteins in all methods) be shared among samples (FIGS. 4E-4G), complimenting the heat map of all quantified proteins in FIG. 4A. Using a PCA plot, clustering between samples can be observed using PC 1 and PC 2 (FIG. 4H). Interestingly, the UC-based sample clusters furthest away from all other samples due to the differences in number of quantified proteins and their relative abundances. GO Term analysis shows the majority of proteins identified in each sample are associated with EVs, acting as an indicator that the developed methods are suitable for EV enrichment (FIG. 4I).

Altogether, the present chromatography-based techniques resulted in comparable intact EV enrichment and lipoprotein particle and plasma protein depletion to the established methods with subtle differences. Through intact particle and protein analysis, the inventors determined the use of CaptoCore is a suitable method to use for EV isolation, enrichment, and/or purification from plasma, with the methods being more time-saving and assessable, requiring less equipment, expense, and personnel training than UC-based methods, and can offer a purer EV sample than a standard CL-2B column performing only SEC. For ease of use, the present methods can be scaled up or down to accommodate various sample volumes of interest and various numbers of samples to process simultaneously. The choice of the stationary phase (e.g., CaptoCore 400 or 700) as well as the format (suspension or sandwich column) used for isolation, can be based upon the requirements and capabilities of the downstream assays, where either high purity or high recovery of EV isolation needs to be prioritized. Moreover, based on the underlying separation mechanisms, the developed techniques can be potentially applicable for the fast and simple isolation of virus particles due to their chemical and structural similarities with EVs.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

### REFERENCES

1 Zhang, H. et al. Identification of distinct nanoparticles and subsets of extracellular vesicles by asymmetric flow field-flow fractionation. Nat Cell Biol 20, 332-343, doi: 10.1038/s41556-018-0040-4 (2018).

2 Harding C., H. J., Stahl P. Endocytosis and intracellular processing of transferrin and colloidal gold-transferrin in rat reticulocytes: demonstration of a pathway for receptor shedding. European Journal of Cell Biology 35, 256-263 (1984).

3 Thery, C. et al. Minimal information for studies of extracellular vesicles 2018 (MISEV2018): a position statement of the International Society for Extracellular Vesicles and update of the MISEV2014 guidelines. J Extracell Vesicles 7, 1535750, doi: 10.1080/20013078.2018. 1535750 (2018).

4 Zhang, Q. et al. Supermeres are functional extracellular nanoparticles replete with disease biomarkers and therapeutic targets. Nat Cell Biol 23, 1240-1254, doi: 10.1038/s41556-021-00805-8 (2021).

5 Zhang, Q. et al. Transfer of Functional Cargo in Exomeres. Cell Rep 27, 940-954 e946, doi: 10.1016/j.celrep.2019.01.009 (2019).

6 Comelli, L. et al. Characterization of secreted vesicles from vascular smooth muscle cells. Mol Biosyst 10, 1146-1152, doi: 10.1039/c3mb70544g (2014).

7 Hoshino, A. et al. Extracellular Vesicle and Particle Biomarkers Define Multiple Human Cancers. Cell 182, 1044-1061 e1018, doi: 10.1016/j.cell.2020.07.009 (2020).

8 Shah, R., Patel, T. & Freedman, J. E. Circulating Extracellular Vesicles in Human Disease. N Engl J Med 379, 958-966, doi: 10. 1056/NEJMra1704286 (2018).

9 Urabe, F. et al. Extracellular vesicles as biomarkers and therapeutic targets for cancer. Am J Physiol Cell Physiol 318, C29-C39, doi: 10.1152/ajpcell.00280.2019 (2020).

10 Lu, Y. T., Delijani, K., Mecum, A. & Goldkorn, A. Current status of liquid biopsies for the detection and management of prostate cancer. Cancer Manag Res 11, 5271-5291, doi: 10.2147/CMAR.S170380 (2019).

11 Fais, S. et al. Evidence-Based Clinical Use of Nanoscale Extracellular Vesicles in Nanomedicine. ACS Nano 10, 3886-3899, doi: 10. 1021/acsnano.5b08015 (2016).

12 Brown, B. A. et al. Charge Detection Mass Spectrometry Measurements of Exosomes and other Extracellular Particles Enriched from Bovine Milk. Anal Chem 92, 3285-3292, doi: 10.1021/acs.analchem.9b05173 (2020).

13 Boing, A. N. et al. Single-step isolation of extracellular vesicles by size-exclusion chromatography. J Extracell Vesicles 3, doi: 10.3402/jev.v3.23430 (2014).

14 Blans, K. et al. Pellet-free isolation of human and bovine milk extracellular vesicles by size-exclusion chromatography. J Extracell Vesicles 6, 1294340, doi: 10.1080/20013078.2017. 1294340 (2017).

15 Watson, D. C. et al. Scalable, cGMP-compatible purification of extracellular vesicles carrying bioactive human heterodimeric IL-15/lactadherin complexes. J Extracell Vesicles 7, 1442088, doi: 10.1080/20013078.2018.1442088 (2018).

16 Cheruvanky, A. et al. Rapid isolation of urinary exosomal biomarkers using a nanomembrane ultrafiltration concentrator. Am J Physiol Renal Physiol 292, F1657-1661, doi: 10.1152/ajprenal.00434.2006 (2007).

17 Helwa, I. et al. A Comparative Study of Serum Exosome Isolation Using Differential Ultracentrifugation and Three Commercial Reagents. PLOS One 12, e0170628, doi: 10.1371/journal.pone.0170628 (2017).

18 Petersen, K. E. et al. A review of exosome separation techniques and characterization of B16-F10 mouse melanoma exosomes with AF4-UV-MALS-DLS-TEM. Anal Bioanal Chem 406, 7855-7866, doi: 10. 1007/s00216-014-8040-0 (2014).

19 Clotilde Thery, A. C., Sebastian Amigorena, and Graca Raposo. Isolation and Characterization of Exosomes from Cell Culture Supernatants and Biological Fluids. Protocols in Cell Biology 3.22 (2006).

20 Lasser, C., Eldh, M. & Lotvall, J. Isolation and characterization of RNA-containing exosomes. J Vis Exp, e3037, doi: 10.3791/3037 (2012).

21 Karimi, N. et al. Detailed analysis of the plasma extracellular vesicle proteome after separation from lipoproteins. Cell Mol Life Sci 75, 2873-2886, doi: 10.1007/s00018-018-2773-4 (2018).

22 Sodar, B. W. et al. Low-density lipoprotein mimics blood plasma-derived exosomes and microvesicles during isolation and detection. Sci Rep 6, 24316, doi: 10.1038/srep24316 (2016).

23 Mahley, R. W., Innerarity, T. L., Rall, S. C. & Weisgraber, K. H. Plasma lipoproteins: apolipoprotein structure and function. Journal of Lipid Research 25, 1277-1294, doi: 10.1016/s0022-2275(20)34443-6 (1984).

24 Elena V. Orlova, M. B. S., Wah Chiu, Hiro Mowri, Louis C. Smith, Antonio M. Gotto Jr. Three-dimensional structure of low density lipoproteins by electron cryomicroscopy. Biochemistry 96 (1999).

25 Momen-Heravi, F. et al. Impact of biofluid viscosity on size and sedimentation efficiency of the isolated microvesicles. Front Physiol 3, 162, doi: 10.3389/fphys.2012.00162 (2012).

26 Momen-Heravi, F. et al. Current methods for the isolation of extracellular vesicles. Biol Chem 394, 1253-1262, doi: 10.1515/hsz-2013-0141 (2013).

27 Anderson, N. L. & Anderson, N. G. The human plasma proteome: history, character, and diagnostic prospects. Mol Cell Proteomics 1, 845-867, doi: 10.1074/mcp.r200007-mcp200 (2002).

28 Welton, J. L., Webber, J. P., Botos, L. A., Jones, M. & Clayton, A. Ready-made chromatography columns for extracellular vesicle isolation from plasma. J Extracell Vesicles 4, 27269, doi: 10.3402/jev.v4.27269 (2015).

29 Takov, K., Yellon, D. M. & Davidson, S. M. Comparison of small extracellular vesicles isolated from plasma by ultracentrifugation or size-exclusion chromatography: yield, purity and functional potential. J Extracell Vesicles 8, 1560809, doi: 10.1080/20013078.2018. 1560809 (2019).

30 James, K. T. et al. Novel High-throughput Approach for Purification of Infectious Virions. Sci Rep 6, 36826, doi: 10.1038/srep36826 (2016).

31 Zhang, K. & Liu, X. Mixed-mode chromatography in pharmaceutical and biopharmaceutical applications. J Pharm Biomed Anal 128, 73-88, doi: 10.1016/j.jpba.2016.05.007 (2016).

32 Zhao, D. et al. Enterovirus71 virus-like particles produced from insect cells and purified by multistep chromatography elicit strong humoral immune responses in mice. J Appl Microbiol 119, 1196-1205, doi: 10.1111/jam. 12922 (2015).

33 Gould, S. J., Booth, A. M. & Hildreth, J. E. The Trojan exosome hypothesis. Proc Natl Acad Sci USA 100, 10592-10597, doi: 10.1073/pnas. 1831413100 (2003).

34 Welsh, J. A. et al. A simple, high-throughput method of protein and label removal from extracellular vesicle samples. Nanoscale 13, 3737-3745, doi: 10.1039/d0nr07830a (2021).

35 Monguio-Tortajada, M., Galvez-Monton, C., Bayes-Genis, A., Roura, S. & Borras, F. E. Extracellular vesicle isolation methods: rising impact of size-exclusion chromatography. Cell Mol Life Sci 76, 2369-2382, doi: 10.1007/s00018-019-03071-y (2019).

36 Mi, X., Fuks, P., Wang, S. C., Winters, M. A. & Carta, G. Protein Adsorption on Core-shell Particles: Comparison of Capto Core 400 and 700 Resins. J Chromatogr A 1651, 462314, doi:10.1016/j.chroma.2021.462314 (2021).

37 Sanchez-Trasvina, C. et al. Structure and functional properties of Capto Core 700 core-shell particles. J Chromatogr A 1621, 461079, doi: 10.1016/j.chroma.2020.461079 (2020).

38 Tian, Y. et al. Quality and efficiency assessment of six extracellular vesicle isolation methods by nano-flow cytometry. J Extracell Vesicles 9, 1697028, doi: 10.1080/20013078.2019.1697028 (2020).

39 Graca Raposo, H. W. N., Willem Stoorvogel, Richtje Leijendekker, Clifford V. Harding, Cornelis J. M. Melief, and Hans J. Geuze*. B Lymphocytes Secrete Antigen-presenting Vesicles. J. Exp. Med. 183, 1161-1172 (1996).

40 Raposo, G. & Stoorvogel, W. Extracellular vesicles: exosomes, microvesicles, and friends. J Cell Biol 200, 373-383, doi: 10.1083/jcb.201211138 (2013).

41 Welsh, J. A. & Jones, J. C. Small Particle Fluorescence and Light Scatter Calibration Using FCMPASS Software. Curr Protoc Cytom 94, e79, doi: 10.1002/cpcy.79 (2020).

42 Welsh, J. A., Jones, J. C. & Tang, V. A. Fluorescence and Light Scatter Calibration Allow Comparisons of Small Particle Data in Standard Units across Different Flow Cytometry Platforms and Detector Settings. Cytometry A 97, 592-601, doi: 10.1002/cyto.a.24029 (2020).

43 Welsh, J. A. et al. FCMPASS Software Aids Extracellular Vesicle Light Scatter Standardization. Cytometry A 97, 569-581, doi: 10.1002/cyto.a.23782 (2020).

44 Daaboul, G. G. et al. Digital Detection of Exosomes by Interferometric Imaging. Sci Rep 6, 37246, doi: 10.1038/srep37246 (2016).

45 Brennan, K. et al. A comparison of methods for the isolation and separation of extracellular vesicles from protein and lipid particles in human serum. Sci Rep 10, 1039, doi: 10.1038/s41598-020-57497-7 (2020).

46 Rembert Pieper, Q. S. & Christine L. Gatlin, S.-T. H., N. Leigh Anderson, Sandra Steiner. Multi-component immunoaffinity subtraction chromatography: An innovative step towards a comprehensive survey of the human plasma proteome. Proteomics 3 (2003).

47 Dovrat, S. et al. 14-3-3 and beta-catenin are secreted on extracellular vesicles to activate the oncogenic Wnt pathway. Mol Oncol 8, 894-911, doi: 10.1016/j.molonc.2014.03.011 (2014).

48 Kugeratski, F. G. et al. Quantitative proteomics identifies the core proteome of exosomes with syntenin-1 as the highest abundant protein and a putative universal biomarker. Nat Cell Biol 23, 631-641, doi: 10.1038/s41556-021-00693-y (2021).

49 Henne, W. M., Buchkovich, N. J. & Emr, S. D. The ESCRT pathway. Dev Cell 21, 77-91, doi: 10.1016/j.devcel.2011.05.015 (2011).

50 Schwenk, J. M. et al. The Human Plasma Proteome Draft of 2017: Building on the Human Plasma PeptideAtlas from Mass Spectrometry and Complementary Assays. J Proteome Res 16, 4299-4310, doi: 10.1021/acs.jproteome.7b00467 (2017).

51 Brittain, G. C. t. et al. A Novel Semiconductor-Based Flow Cytometer with Enhanced Light-Scatter Sensitivity for the Analysis of Biological Nanoparticles. Sci Rep 9, 16039, doi: 10.1038/s41598-019-52366-4 (2019).

52 Emma J.K. Kowal, D. T.-O., Aviv Regev, George M. Church. Extracellular Vesicle Isolation and Analysis by The western Blotting. Methods in Molecular Biology 1660, 10, doi: 10.1007/978-1-4939-7253-1_12 (2017).

53 Kostas, J. C., Gregus, M., Schejbal, J., Ray, S. & Ivanov, A. R. Simple and Efficient Microsolid-Phase Extraction Tip-Based Sample Preparation Workflow to Enable Sensitive Proteomic Profiling of Limited Samples (200 to 10,000 Cells). J Proteome Res 20, 1676-1688, doi: 10.1021/acs.jproteome.0c00890 (2021).

54 Perez-Riverol, Y. et al. The PRIDE database and related tools and resources in 2019: improving support for quantification data. Nucleic Acids Res 47, D442-D450, doi: 10.1093/nar/gkyl106 (2019).

55 Chen, C. et al. TBtools: An Integrative Toolkit Developed for Interactive Analyses of Big Biological Data. Mol Plant 13, 1194-1202, doi: 10.1016/j.molp.2020.06.009 (2020).

56 Pathan, M. et al. FunRich: An open access standalone functional enrichment and interaction network analysis tool. Proteomics 15, 2597-2601, doi: 10.1002/pmic.201400515 (2015).

57 Metsalu, T. & Vilo, J. ClustVis: a web tool for visualizing clustering of multivariate data using Principal Component Analysis and heatmap. Nucleic Acids Res 43, W566-570, doi: 10.1093/nar/gkv468 (2015).

What is claimed is:

1. A method of isolating and/or purifying a nanoparticulate biomaterial, the method comprising the steps of:
(a) providing a sample comprising the nanoparticulate biomaterial and a multimodal chromatography resin, wherein the sample comprises said nanoparticulate biomaterial suspended in an aqueous liquid; and
(b) contacting the sample with the multimodal chromatography resin for an incubation period, and;
(c) separating the nanoparticulate biomaterial from the multimodal chromatography resin, whereby the nanoparticulate biomaterial is isolated and/or purified from other components of the sample.

2. The method of claim 1, wherein the nanoparticulate biomaterial is selected from the group consisting of extracellular vesicles and particles (EVP), viruses, viral vectors, pseudoviruses, virus-like particles, polymeric nanoparticles, lipidic nanoparticles, artificial lipid membrane vesicles, and nanoparticulate drug carriers.

3. The method of claim 2, wherein the nanoparticulate biomaterial is EVP comprising exomeres, supermeres, exosomes, and/or microvesicles.

4. The method of claim 1, wherein the nanoparticulate biomaterial has an average particle size in a range from about 50 nm to about 100 nm, or from about 100 nm to about 150 nm, or from about 100 nm to about 200 nm, about 200 nm or less, from about 200 nm to about 1000 nm, or more than 1000 nm.

5. The method of claim 1, wherein the sample is a biological fluid; plasma;
interstitial fluid; cerebrospinal fluid; saliva; cell culture medium; homogenized biological cells, tissue, or extracellular matrix; a pharmaceutical composition; a vaccine composition; or aerosol droplets or breath condensate; tear fluid; tissue aspirate, sputum; nasal fluid; lavage; artificially prepared emulsion, micelles, lipid droplets, or liposomes; or a tissue homogenate from a mammal, plant, mushroom, or alga.

6. The method of claim 1, wherein the multimodal chromatography resin comprises porous resin particles having one or more functional groups selectively disposed inside the porous resin particles.

7. The method of claim 6, wherein the functional groups are selected from ionic groups, hydrophobic groups, hydrophilic groups, amphiphilic groups, polar uncharged groups, non-polar groups, affinity-based groups, octylamine, and N-benzyl, N-methyl ethanolamine.

8. The method of claim 6, wherein the multimodal chromatography resin is capable of separating said nanoparticulate biomaterial from other components of the sample by at least a first mode and a second mode, wherein the first mode is size exclusion chromatography, and wherein the second mode is different from size exclusion chromatography.

9. The method of claim 8, wherein the second mode is ion exchange chromatography, hydrophobic interaction chromatography, reversed phase chromatography, or affinity chromatography.

10. The method of claim 9, wherein the ion exchange chromatography is anion exchange or cation exchange chromatography.

11. The method of claim 6, wherein the porous resin particles comprise pores having an average size of about 19 nm, about 50 nm, or about 72 nm.

12. The method of claim 1, wherein the step of contacting is performed using a suspension of particles of said multimodal chromatography resin in an aqueous medium.

13. The method of claim 12, wherein the suspension is mixed during the incubation period.

14. The method of claim 1, wherein the incubation period is long enough to attain binding equilibrium of undesired components of the sample with the functional groups inside the porous resin particles of the multimodal chromatography resin.

15. The method of claim 12, wherein the step of separating includes centrifugation or filtration of the suspension and harvesting the isolated and/or purified nanoparticulate biomaterial from the supernatant or filtrate.

16. The method of claim 1, wherein the step of contacting is performed using a bed of said multimodal chromatography resin, the bed disposed in a chromatography column, and wherein the step of separating is performed by eluting fractions from the column, some of said fractions comprising the isolated and/or purified nanoparticulate biomaterial.

17. The method of claim 16, wherein the chromatography column is a sandwich column comprising the bed of said multimodal chromatography resin and a second bed comprising a second chromatography resin, wherein the multimodal chromatography resin and the second chromatography resin are capable of different modes of separation.

18. The method of claim 17, wherein the multimodal chromatography resin performs size exclusion and anion exchange and the second chromatography resin performs size exclusion.

19. The method of claim 17, wherein the multimodal chromatography resin is disposed above the second chromatography resin in the column.

20. The method of claim 16, wherein the chromatography column is a multilayer column comprising the bed (layer) of said multimodal chromatography resin and at least two other beds (layers) comprising a other chromatography resins, wherein the multimodal chromatography resin and the other chromatography resins are capable of different two or more modes of separation.

21. The method of claim 1, wherein the method does not comprise ultracentrifugation.

22. The method of claim 1, wherein the nanoparticulate biomaterial is separated from other biomaterial present in the sample.

23. The method of claim 22, wherein the other biomaterial is protein or other particulate material.

24. The method of claim 1, wherein the nanoparticulate biomaterial is fractionated to yield one or more isolated and/or purified subpopulations.

25. The method of claim 24, wherein the nanoparticulate biomaterial that is isolated and/or purified is EVP, and the other biomaterial is plasma protein, albumin, immunoglobulin, alpha-2 macroglobulin, apolipoprotein B100, apolipoprotein A1, haptoglobin, serotransferrin, and/or alpha-1-antitrypsin.

26. The method of claim 23, wherein the isolated and/or purified EVP are enriched in CD9, annexin A2, and/or 14-3-3 zeta/delta compared to the sample.

27. The method of claim 1, further comprising analyzing the separated nanoparticulate biomaterial using a method selected from TEM, BCA, WB, TRPS, nFCM, or nLC-MS/MS.

28. A chromatography column configured for performing the method of claim 1.

29. The chromatography column of claim 28, comprising a multimodal chromatography resin and a size exclusion chromatography resin, and optionally one or more other chromatography resins, wherein the chromatography column is configured as a sandwich column or a multilayered column.

30. A kit for isolating and/or purifying a nanoparticulate biomaterial, the kit comprising:
a multimodal chromatography resin or a column comprising a multimodal chromatography resin; and
instructions for carrying out the method of claim 1.

31. A method of diagnosing a disease or medical condition, the method comprising the steps of:
(a) providing a sample from a subject suspected of having the disease or medical condition and a chromatography column comprising a multimodal chromatography resin and a size exclusion chromatography resin, and optionally one or more other chromatography resins, wherein the chromatography column is configured as a sandwich column or a multilayered column;
(b) performing the method of claim 1, whereby an isolated and/or purified nanoparticulate biomaterial is obtained; and
(c) analyzing the isolated and/or purified nanoparticulate biomaterial to diagnose the disease or medical condition.

32. The method of claim 31, further comprising:
(d) treating the disease or medical condition.
